(19) United States
(12) Patent Application Publication
(10) Pub. No.: US 2025/0134394 A1
(43) Pub. Date: May 1, 2025

(54) HEARTBEAT AND RESPIRATION DETECTION DEVICE AND HEARTBEAT AND RESPIRATION DETECTION PROGRAM

(71) Applicant: NISSHINBO SINGAPORE PTE. LTD., Singapore Land Tower (SG)

(72) Inventors: Kazuo Saito, Singapore Land Tower (SG); Kengo Tsushima, Singapore Land Tower (SG); Minori Fujii, Singapore Land Tower (SG)

(73) Assignee: NISSHINBO SINGAPORE PTE. LTD., Singapore Land Tower (SG)

(21) Appl. No.: 18/690,762

(22) PCT Filed: Sep. 30, 2022
(86) PCT No.: PCT/JP2022/036795

§ 371 Date: Mar. 11, 2024

(30) Foreign Application Priority Data
Oct. 4, 2021 (JP) 2021-163311

(51) Int. Cl. A61B 5/0205; A61B 8/02

(52) U.S. Cl. CPC A61B 5/0205; A61B 8/02

(57) ABSTRACT

In this disclosure, at heartbeat detection, a frequency component caused by a micro vibration of heartbeat is extracted over a predefined heartbeat observation time window, an amplitude peak is extracted from the frequency component caused by the micro vibration of the heartbeat, and one characteristic micro vibration out of a plurality of characteristic micro vibrations in one heartbeat is extracted. On the other hand, at respiration detection, positive and negative frequency components caused by the micro vibration of the heartbeat are extracted, complex multiplication of the positive and negative frequency components is performed, and from complex multiplication of the positive and negative frequency components, a respiration phase change caused by a micro vibration of respiration is extracted, with a heartbeat phase change caused by the micro vibration of the heartbeat removed.

### TECHNICAL FIELD

This disclosure relates to a technique of calculating a heart rate and a respiration rate while enabling the alleviation of burdens on nurses and infection risk reduction based on radar signals or ultrasound signals reflecting off a body surface.

### BACKGROUND ART

Patent Literature 1 and the like disclose a technique of calculating a heart rate and a respiration rate while enabling the alleviation of burdens on nurses and infection risk reduction based on radar signals reflecting off a body surface.

FIG. 1 illustrates an outline of heartbeat and respiration detection processing of the prior art. From radar signals reflecting off a body surface, a spectrogram S that shows a temporal change of each frequency component is calculated. From the spectrogram S, a direct current (DC) component including an extremely low frequency component is extracted. About once or twice in 10 seconds, amplitude peaks caused by a micro vibration of respiration are extracted. Based on a time interval of the respiration amplitude peaks, a respiration rate is calculated. About three times between the respiration amplitude peaks, amplitude peaks caused by a micro vibration of heartbeat are extracted. Based on a time interval of the heartbeat amplitude peaks, a heart rate is calculated.

### CITATION LIST

### Patent Literature

Patent Literature 1: JP-A-2019-129996

### SUMMARY OF INVENTION

### Technical Problem

FIG. 1 illustrates the DC component including an extremely low frequency component being extracted from the spectrogram S. Since disturbances, such as louvers of air conditioners, curtains swayed by the wind and the movements of nurses, are mainly included in this extremely low frequency component and are close to a frequency component caused by the micro vibration of the respiration, it is difficult to ensure the improvement of robustness and the reduction of the effects of disturbances. Then, since the heartbeat amplitude peaks are small compared with the respiration amplitude peaks, it is difficult to separate the heartbeat amplitude peaks from the respiration amplitude peaks. Furthermore, if a harmonic frequency that is three or four times a respiration frequency is approximately equal to a heartbeat frequency, it is difficult to separate the heartbeat amplitude peaks from the respiration amplitude peaks. Even if the heartbeat amplitude peaks can be separated from the respiration amplitude peaks, problems described in FIG. 2 and FIG. 3 exist.

FIG. 2 illustrates a problem of heartbeat detection processing of the prior art. In one heartbeat, amplitude peaks of a first sound and a second sound are present. Accordingly, it is difficult to avoid a possibility that a heart rate (four times of (1), (2), (3), and (4)) which is twice a true heart rate (two times of (1) and (3) or (2) and (4)) is calculated. When A (bpm) is a detection object as a heart rate, 2A (bpm) cannot be a detection object as a heart rate, and therefore, it is difficult to extend a detection range of a heart rate.

FIG. 3 illustrates a problem of respiration detection processing of the prior art. Although, in one respiration, the movements of a chest and an abdomen are out of synchronization in some cases, the movements of the chest and the abdomen are synthesized without being discriminated in a radar signal. Accordingly, it is difficult to avoid a possibility that a respiration rate (four times of (1), (2), (3), and (4) of respiration, synthesis) which is twice a true respiration rate (two times of (1) and (2) of respiration, chest or respiration, abdomen) is calculated. When B (bpm) is a detection object as a respiration rate, 2B (bpm) cannot be a detection object as a respiration rate, and therefore, it is difficult to extend a detection range of a respiration rate.

Therefore, in order to solve the above-described problems, an object of this disclosure is to ensure the improvement of robustness, the reduction of the effects of disturbances, the separation of heartbeat and respiration, the avoidance of multiple times counting, and the extension of a detection range when calculating a heart rate and a respiration rate while enabling the alleviation of burdens on nurses and infection risk reduction based on a radar signal (an ultrasound signal may be included) reflecting off a body surface.

### Solution to Problem

In order to solve the above-described problem of heartbeat detection, a frequency component caused by a micro vibration of heartbeat is extracted from a radar signal or an ultrasound signal reflecting off a body surface over a predefined heartbeat observation time window. Then, amplitude peaks are extracted from the frequency component caused by the micro vibration of the heartbeat, and one characteristic micro vibration out of a plurality of characteristic micro vibrations in one heartbeat is extracted.

Specifically, this disclosure is a heartbeat and respiration detection device including: a heartbeat component extraction unit that extracts a frequency component caused by a micro vibration of heartbeat from a radar signal or an ultrasound signal reflecting off a body surface over a predefined heartbeat observation time window; a heartbeat peak extraction unit that extracts an amplitude peak from the frequency component caused by the micro vibration of the heartbeat and extracts one characteristic micro vibration out of a plurality of characteristic micro vibrations in one heartbeat; and a heart rate calculation unit that calculates a heart rate based on a time interval of the amplitude peaks or a count of the amplitude peaks extracted within a predetermined time.

With this configuration, since the frequency component caused by the micro vibration of the heartbeat is extracted, the improvement of robustness and the reduction of the effects of disturbances can be ensured. Then, since one characteristic micro vibration out of the plurality of characteristic micro vibrations in one heartbeat is extracted, the avoidance of multiple times counting and the extension of a detection range can be ensured. Furthermore, since data that serves as a basis for calculating the heart rate is different from data that serves as a basis for calculating a respiration rate (described below), the separation of heartbeat and respiration can be ensured.

In addition, this disclosure is the heartbeat and respiration detection device in which the heartbeat component extraction unit extracts the frequency component caused by the micro vibration of the heartbeat over the predefined heartbeat observation time window including a plurality of characteristic micro vibrations in one heartbeat.

With this configuration, since the plurality of characteristic micro vibrations in one heartbeat are synthesized and output at the time of extracting the frequency component caused by the micro vibration of the heartbeat, the avoidance of multiple times counting can be approximately completely ensured. However, depending on a time width of the predefined heartbeat observation time window, only one characteristic micro vibration in one heartbeat may be included in the predefined heartbeat observation time window.

In addition, this disclosure is the heartbeat and respiration detection device in which the heartbeat peak extraction unit extracts a maximum amplitude peak of the amplitude peaks from the frequency component caused by the micro vibration of the heartbeat in a predefined peak detection time window.

With this configuration, even when only one characteristic micro vibration in one heartbeat is included in the predefined heartbeat observation time window, only the maximum amplitude peak is extracted in the predefined peak detection time window, and therefore, the avoidance of multiple times counting can be approximately completely ensured. However, depending on a time width of the predefined peak detection time window, an upper limit and a lower limit of the heart rate are narrowed to some extent.

In addition, this disclosure is the heartbeat and respiration detection device in which the heartbeat peak extraction unit moves the predefined peak detection time window so as to extract the maximum amplitude peak in a time domain excluding vicinities of both ends of the predefined peak detection time window.

Since in this configuration, one characteristic micro vibration in one heartbeat is made to avoid straddling an adjacent predefined peak detection time window, and therefore, the upper limit of the heart rate can be extended.

In addition, this disclosure is the heartbeat and respiration detection device in which the heart rate calculation unit calculates a heart rate based on a time interval of the maximum amplitude peaks extracted in an adjacent or non-adjacent predefined peak detection time window.

With this configuration, the lower limit of the heart rate can be extended considering that the plurality of characteristic micro vibrations in one heartbeat are included in predefined peak detection time windows that lie at intervals.

In addition, this disclosure is the heartbeat and respiration detection device in which the heart rate calculation unit calculates an average heart rate with increasing a weight of a time interval of the amplitude peaks as an amplitude value of the amplitude peak increases.

With this configuration, since the time interval of the heartbeat amplitude peaks is weighted according to the magnitudes of the amplitude values of the heartbeat amplitude peaks, the calculation of the heart rate can be performed with high accuracy.

In addition, this disclosure is the heartbeat and respiration detection device in which the heart rate calculation unit calculates a heart rate based on clustering of two-dimensional data constituted of a time interval of the amplitude peaks and a weight of the time interval with increasing the weight of the time interval of the amplitude peaks as an amplitude value of the amplitude peak increases.

With this configuration, since the weight of the time interval of the heartbeat amplitude peaks is calculated according to the magnitudes of the amplitude values of the heartbeat amplitude peaks, the calculation of the heart rate can be performed with high accuracy.

In order to solve the above-described problem of respiration detection, positive and negative frequency components caused by the micro vibration of the heartbeat is extracted from the radar signal or the ultrasound signal reflecting off the body surface, and complex multiplication of the positive and negative frequency components is performed. Then, from complex multiplication of the positive and negative frequency components, a respiration phase change caused by a micro vibration of respiration is extracted, with a heartbeat phase change caused by the micro vibration of the heartbeat removed.

Specifically, this disclosure is a heartbeat and respiration detection device including: a heartbeat component multiplication unit that extracts positive and negative frequency components caused by a micro vibration of heartbeat from a radar signal or an ultrasound signal reflecting off a body surface and performs complex multiplication of the positive and negative frequency components; a respiration phase extraction unit that extracts a respiration phase change caused by a micro vibration of respiration, with a heartbeat phase change caused by a micro vibration of heartbeat removed, from complex multiplication of the positive and negative frequency components; and a respiration rate calculation unit that calculates a respiration rate based on a frequency component of the respiration phase change.

With this configuration, since the frequency component caused by the micro vibration of the heartbeat is extracted, the improvement of robustness and the reduction of the effects of disturbances can be ensured. Then, since the respiration rate is calculated based on the frequency component of the respiration phase change with the heartbeat phase change removed, the avoidance of multiple times counting and the extension of a detection range can be ensured in consideration of a reflected signal from a chest without considering a reflected signal from an abdomen. Furthermore, since the data that serves as a basis for calculating the respiration rate is different from the data that serves as a basis for calculating the heart rate (described above), the separation of respiration and heartbeat can be ensured.

In addition, this disclosure is the heartbeat and respiration detection device in which the respiration phase extraction unit extracts amplitude peaks caused by a micro vibration of heartbeat from complex multiplication of the positive and negative frequency components and extracts the respiration phase change at the amplitude peak while applying zero padding between the amplitude peaks without extracting the respiration phase change.

With this configuration, in consideration of the fluctuation of the heartbeat, information on the respiration phase change is used at heartbeat amplitude peak time while the information on the respiration phase change is not used between the heartbeat amplitude peaks, and therefore, the calculation of the respiration rate can be performed with high accuracy.

In addition, this disclosure is the heartbeat and respiration detection device in which the respiration rate calculation unit calculates an average respiration rate with increasing a weight of a respiration rate as a maximum peak amplitude of the frequency component of the respiration phase change increases.

With this configuration, since the respiration rate is weighted according to the magnitude of the maximum peak amplitude of the frequency component of the respiration phase change, the calculation of the respiration rate can be performed with high accuracy.

In addition, this disclosure is a heartbeat and respiration detection program for causing a computer to execute each processing step corresponding to each processing unit of the heartbeat and respiration detection device described above.

This configuration allows providing the program having the above-described effects.

### Advantageous Effects of Invention

Thus, this disclosure can ensure the improvement of robustness, the reduction of the effects of disturbances, the separation of heartbeat and respiration, the avoidance of multiple times counting, and the extension of a detection range when calculating a heart rate and a respiration rate while enabling the alleviation of burdens on nurses and infection risk reduction based on a radar signal (an ultrasound signal may be included) reflecting off a body surface.

### BRIEF DESCRIPTION OF DRAWINGS

FIG. 1 is a drawing illustrating an outline of heartbeat and respiration detection processing of the prior art.

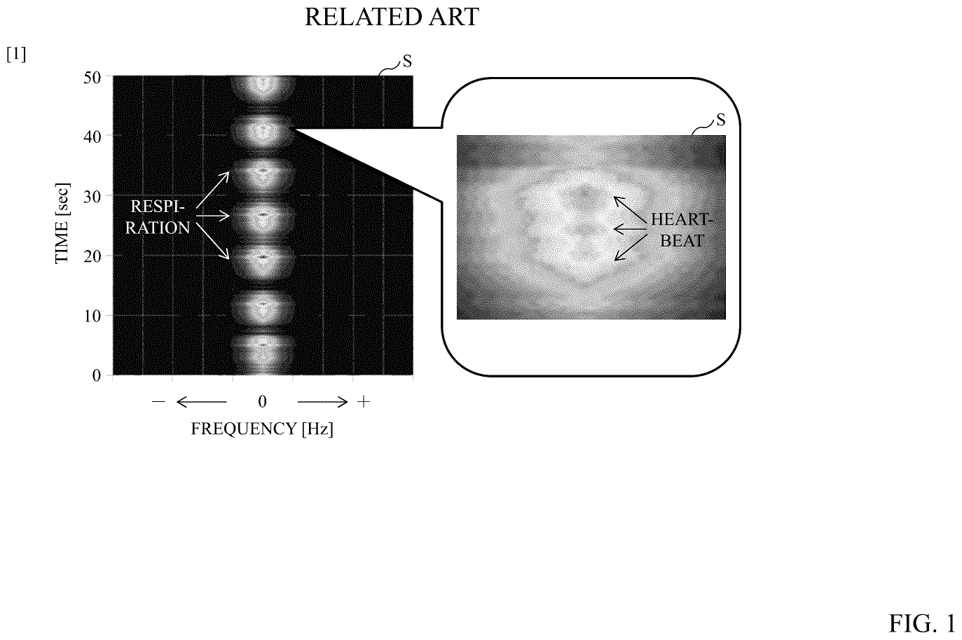

FIG. 2 is a drawing illustrating a problem of heartbeat detection processing of the prior art.

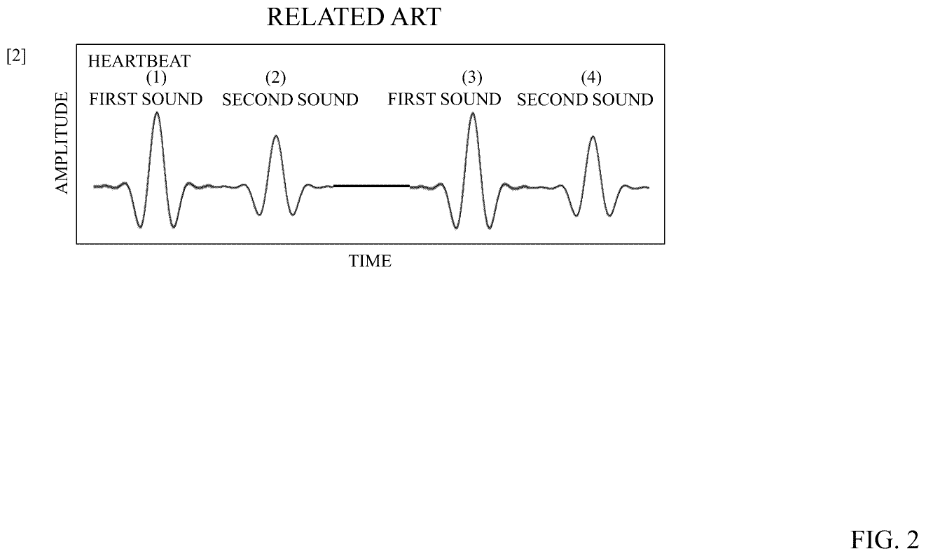

FIG. 3 is a drawing illustrating a problem of respiration detection processing of the prior art.

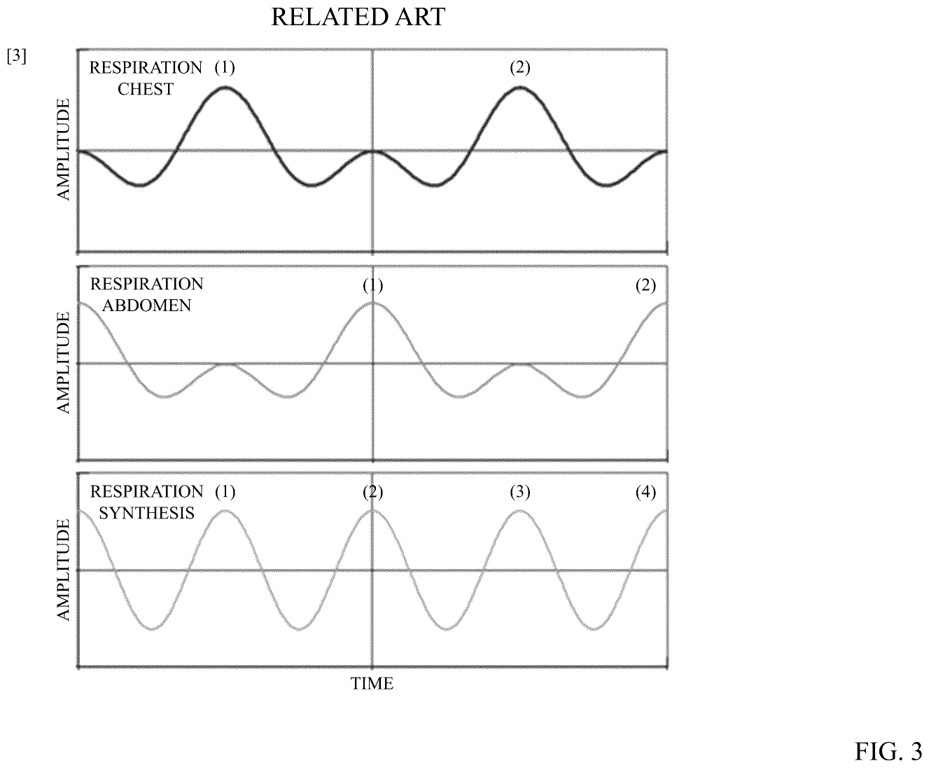

FIG. 4 is a diagram illustrating a configuration of a heartbeat and respiration detection device of this disclosure.

FIG. 5 is a drawing illustrating an outline of heartbeat and respiration detection processing of this disclosure.

FIG. 6 is a diagram illustrating a procedure of heartbeat detection processing of this disclosure.

FIG. 7 is a drawing illustrating a specific example of heartbeat component extraction processing of this disclosure.

FIG. 8 is a drawing illustrating a specific example of first heartbeat detection processing of this disclosure.

FIG. 9 is a drawing illustrating a specific example of the first heartbeat detection processing of this disclosure.

FIG. 10 is a drawing illustrating a specific example of the first heartbeat detection processing of this disclosure.

FIG. 11 is a drawing illustrating a specific example of second heartbeat detection processing of this disclosure.

FIG. 12 is a drawing illustrating a specific example of third heartbeat detection processing of this disclosure.

FIG. 13 is a drawing illustrating a specific example of the third heartbeat detection processing of this disclosure.

FIG. 14 is a diagram illustrating a procedure of respiration detection processing of this disclosure.

FIG. 15 is a drawing illustrating a principle of the respiration detection processing of this disclosure.

FIG. 16 is a drawing illustrating the principle of the respiration detection processing of this disclosure.

FIG. 17 is a drawing illustrating the principle of the respiration detection processing of this disclosure.

FIG. 18 is a drawing illustrating a specific example of the respiration detection processing of this disclosure.

FIG. 19 is a drawing illustrating a result of the heartbeat detection processing of this disclosure.

FIG. 20 is a drawing illustrating the result of the heartbeat detection processing of this disclosure.

FIG. 21 is a drawing illustrating a result of the respiration detection processing of this disclosure.

FIG. 22 is a drawing illustrating the result of the respiration detection processing of this disclosure.

FIG. 23 is a drawing illustrating the result of the respiration detection processing of this disclosure.

### DESCRIPTION OF EMBODIMENTS

Embodiments of this disclosure will be described by referring to the accompanying drawings. The embodiments described below are examples of the implementation of this disclosure, and this disclosure is not limited to the following embodiments.

(Configuration of Heartbeat and Respiration Detection Device of this Disclosure)

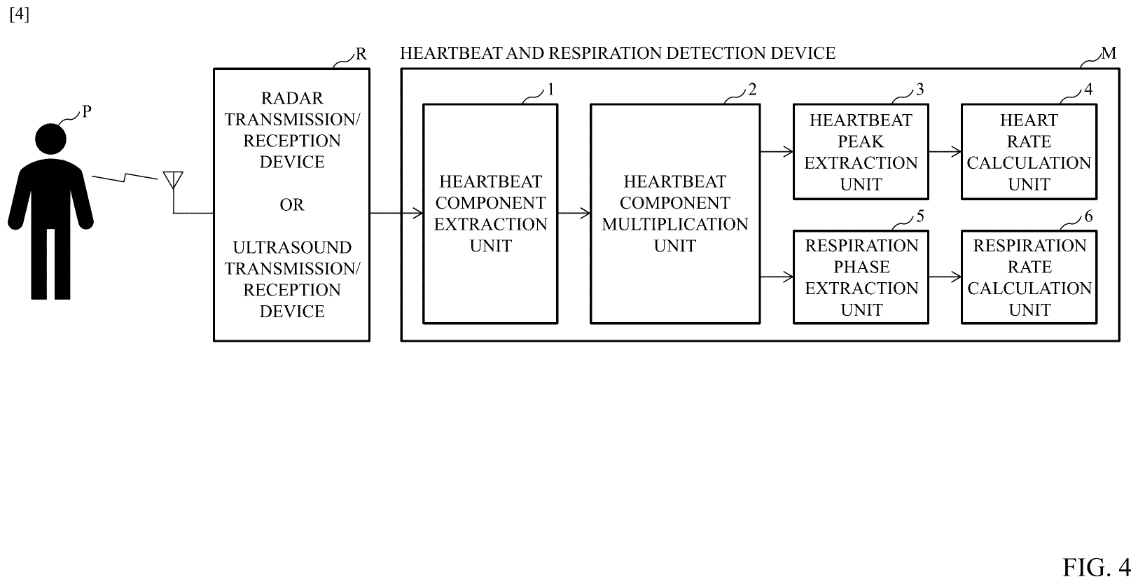

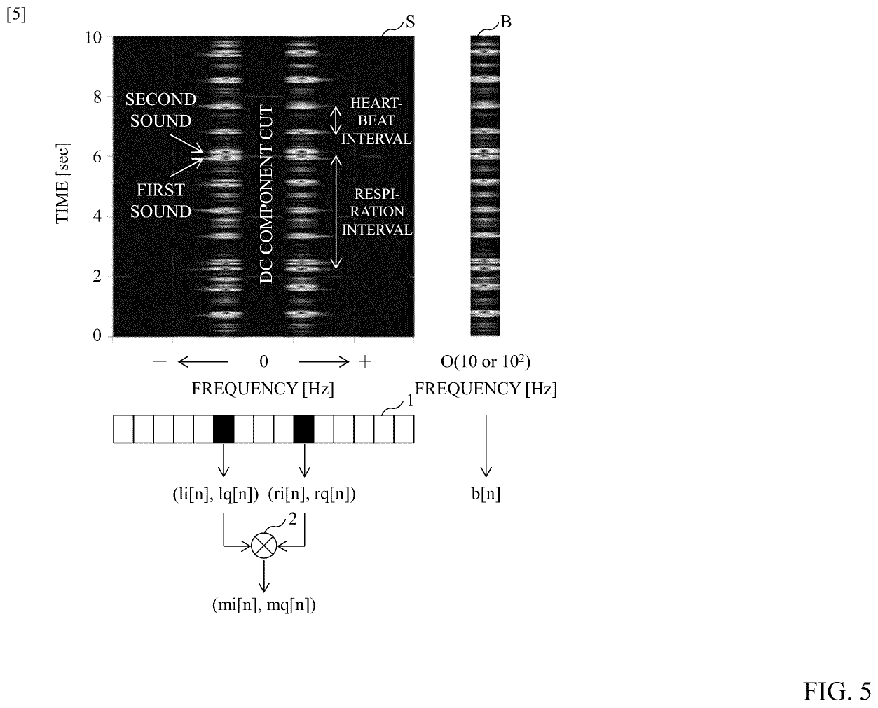

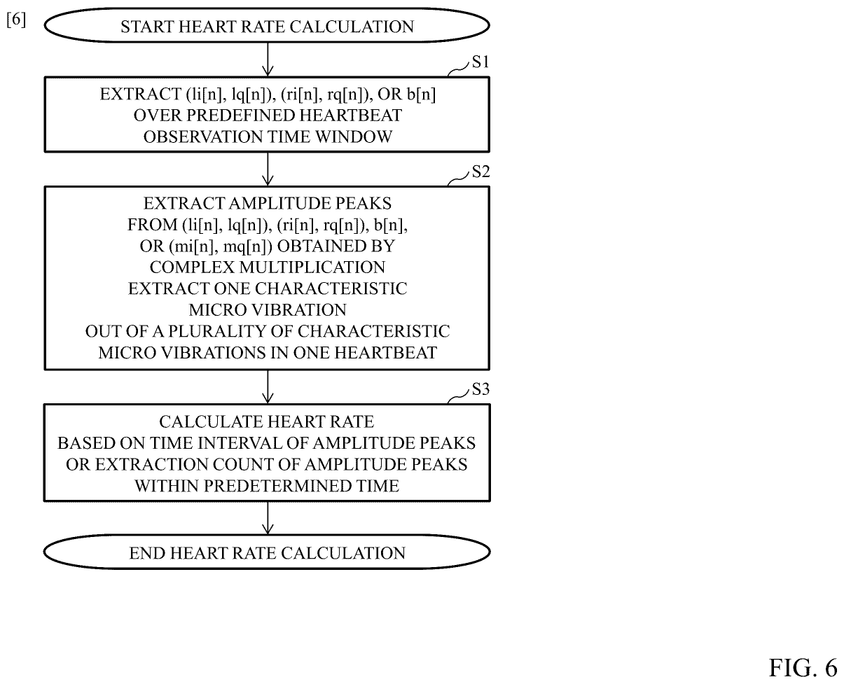

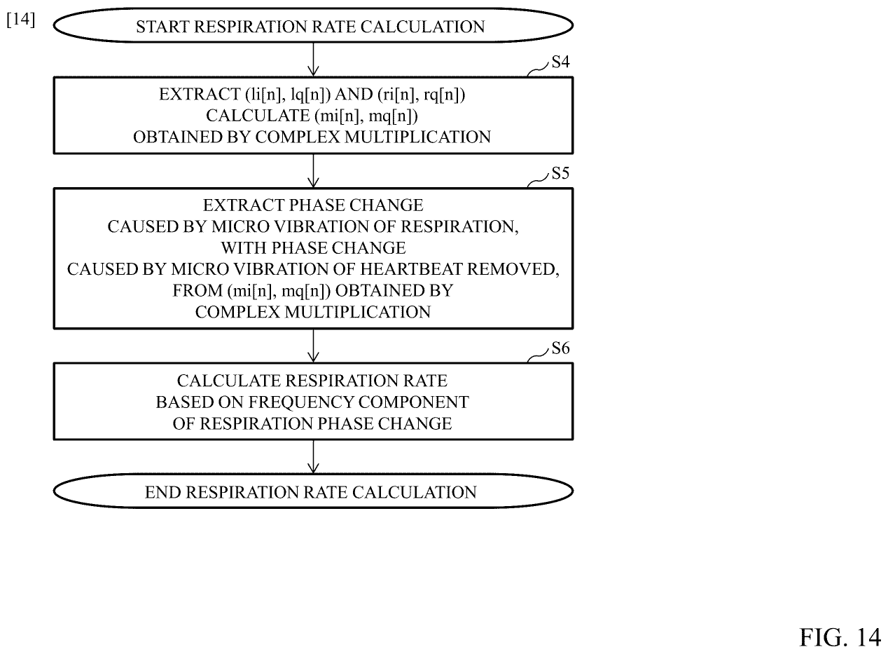

FIG. 4 illustrates a configuration of a heartbeat and respiration detection device of this disclosure. FIG. 5 illustrates an outline of heartbeat and respiration detection processing of this disclosure. A heartbeat and respiration detection device M is equipped with a heartbeat component extraction unit 1, a heartbeat component multiplication unit 2, a heartbeat peak extraction unit 3, a heart rate calculation unit 4, a respiration phase extraction unit 5, and a respiration rate calculation unit 6 and can be realized by installing a heartbeat and respiration detection program illustrated in FIG. 6 and FIG. 14 on a computer.

A radar transmission/reception device R or an ultrasound transmission/reception device R transmits a radar signal or an ultrasound signal (carrier wave band) with which a body surface of a patient P is irradiated, receives the radar signal or the ultrasound signal (carrier wave band) reflecting off the body surface of the patient P, and converts the received radar signal or ultrasound signal to a baseband to output it. A radar method or an ultrasound method may be any of a CW method, an FMCW method, a standing wave method, and other methods. The radar signal or the ultrasound signal (carrier wave band) has a wavelength on the order of 1 mm to 10 mm, which is equal to on the order of a micro vibration width of the body surface of the patient P.

The heartbeat component extraction unit 1 extracts one frequency component or both frequency components of positive and negative frequency components (on the order of ±10 or 102 Hz) caused by a micro vibration of heartbeat from the radar signal or the ultrasound signal (I/Q complex signal of the baseband) reflecting off the body surface of the patient P. Alternatively, the heartbeat component extraction unit 1 extracts the frequency component (on the order of +10 or 10² Hz) caused by the micro vibration of the heartbeat from the radar signal or the ultrasound signal (real number signal of the baseband) reflecting off the body surface of the patient P. The heartbeat component multiplication unit 2 performs complex multiplication of the positive and negative frequency components (on the order of ±10 or 10² Hz) caused by the micro vibration of the heartbeat.

Here, the heartbeat component extraction unit 1 calculates a spectrogram S that shows a temporal change of each frequency component from the radar signal or the ultrasound signal (I/Q complex signal of the baseband) reflecting off the body surface of the patient P. Then, from the spectrogram S, a direct current (DC) component including an extremely low frequency component is removed, and the positive and negative frequency components (on the order of ±10 or 10² Hz) caused by the micro vibration of the heartbeat are extracted. Alternatively, the heartbeat component extraction unit 1 calculates a band-pass filter result B in the frequency component caused by the micro vibration of the heartbeat from the radar signal or the ultrasound signal (real number signal of the baseband) reflecting off the body surface of the patient P. Then, in the band-pass filter result B, approximately similarly to the spectrogram S, the frequency component (on the order of +10 or 10² Hz) caused by the micro vibration of the heartbeat is extracted.

In FIG. 5, about once or twice in 2 seconds, small amplitude peaks caused by the micro vibration of the heartbeat are extracted, and close amplitude peaks of a first sound and a second sound in one heartbeat are extracted. Then, although about once or twice in 4 seconds, large amplitude peaks caused by the micro vibration of the respiration are extracted, a harmonic frequency that is three or four times a respiration frequency may be approximately equal to a heartbeat frequency.

Here, the heartbeat component extraction unit 1 extracts (ri[n], rq[n]) (r is a positive frequency, i and q are i and q components, respectively, and n is time) as a positive frequency component caused by the micro vibration of the heartbeat and extracts (li[n], lq[n]) (l is a negative frequency, i and q are i and q components, respectively, and n is time) as a negative frequency component caused by the micro vibration of the heartbeat from the radar signal or the ultrasound signal (I/Q complex signal of the baseband) reflecting off the body surface of the patient P. Alternatively, the heartbeat component extraction unit 1 extracts b[n] (b is a pass frequency band of the band-pass filter result B, and n is time) as a frequency component caused by the micro vibration of the heartbeat from the radar signal or the ultrasound signal (real number signal of the baseband) reflecting off the body surface of the patient P. Then, the heartbeat component multiplication unit 2 calculates (mi[n], mq[n])=(ri[n]·li[n]−rq[n]·lq[n], rq[n]·li[n]+ri[n]·lq[n]) as complex multiplication of the positive and negative frequency components.

As illustrated in FIG. 6 to FIG. 13, the heartbeat peak extraction unit 3 and the heart rate calculation unit 4 calculate a heart rate based on the frequency components (ri[n], rq[n]), (li[n], lq[n]), b[n], or (mi[n], mq[n]). As illustrated in FIG. 14 to FIG. 18, the respiration phase extraction unit 5 and the respiration rate calculation unit 6 calculate a respiration rate based on the frequency component (mi[n], mq[n]). First, the calculation of the heart rate is described, and next, the calculation of the respiration rate is described.

(Procedure of Heartbeat Detection Processing of this Disclosure: Heartbeat Component Extraction Processing)

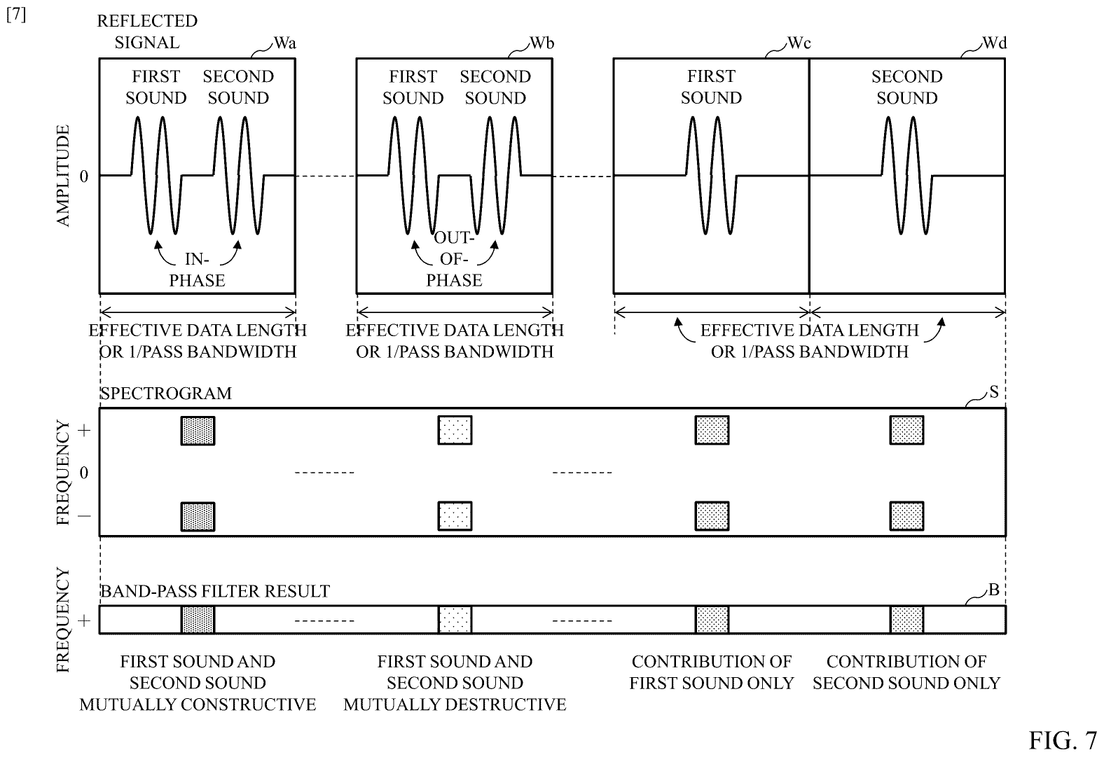

FIG. 6 illustrates a procedure of heartbeat detection processing of this disclosure. The heartbeat component extraction unit 1 extracts the frequency components (ri[n], rq[n]), (li[n], lq[n]), or b[n] over a predefined heartbeat observation time window (Step S1). The heartbeat peak extraction unit 3 extracts amplitude peaks from the frequency components (ri[n], rq[n]), (li[n], lq[n]), b[n], or (mi[n], mq[n]) and extracts one characteristic micro vibration out of a plurality of characteristic micro vibrations in one heartbeat (Step S2). The heart rate calculation unit 4 calculates the heart rate based on a time interval of the amplitude peaks (this embodiment) or a count of the amplitude peaks extracted within a predetermined time (modification) (Step S3). Specifically, the processing from FIG. 7 to FIG. 13 is performed.

Thus, since the frequency component caused by the micro vibration of the heartbeat is extracted, the improvement of robustness and the reduction of the effects of disturbances can be ensured. Then, since one characteristic micro vibration out of the plurality of characteristic micro vibrations in one heartbeat is extracted, the avoidance of multiple times counting and the extension of a detection range can be ensured. Furthermore, since data that serves as a basis for calculating the heart rate is different from data that serves as a basis for calculating a respiration rate (described below), the separation of heartbeat and respiration can be ensured.

Here, in this embodiment, the first sound and the second sound in one heartbeat are employed as the plurality of characteristic micro vibrations in one heartbeat. However, as a modification, three or more characteristic micro vibrations in one heartbeat may be employed depending on an individual difference or animal species of a heartbeat and respiration detection object.

FIG. 7 illustrates a specific example of heartbeat component extraction processing of this disclosure. The heartbeat component extraction unit 1 extracts the frequency components (ri[n], rq[n]), (li[n], lq[n]), or b[n] over a predefined heartbeat observation time window including a plurality of characteristic micro vibrations in one heartbeat (Step S1).

In FIG. 7, a time width of heartbeat observation time windows Wa, Wb, Wc, and Wd is an effective data length or 1/(pass bandwidth of the band-pass filter result B) of the spectrogram S.

In the heartbeat observation time window Wa, the first sound and the second sound in one heartbeat are included in an in-phase state (the phase relationship varies depending on individual differences or animal species). Then, in the spectrogram S, after the first sound and the second sound in one heartbeat are synthesized in a mutually constructive state, the frequency components (ri[n], rq[n]) and (li[n], lq[n]) are extracted. Alternatively, in the band-pass filter result B, after the first sound and the second sound in one heartbeat are synthesized in a mutually constructive state, the frequency component b[n] is extracted. Accordingly, at the time of extracting the frequency components caused by the micro vibration of the heartbeat, the avoidance of multiple times counting can be approximately completely ensured.

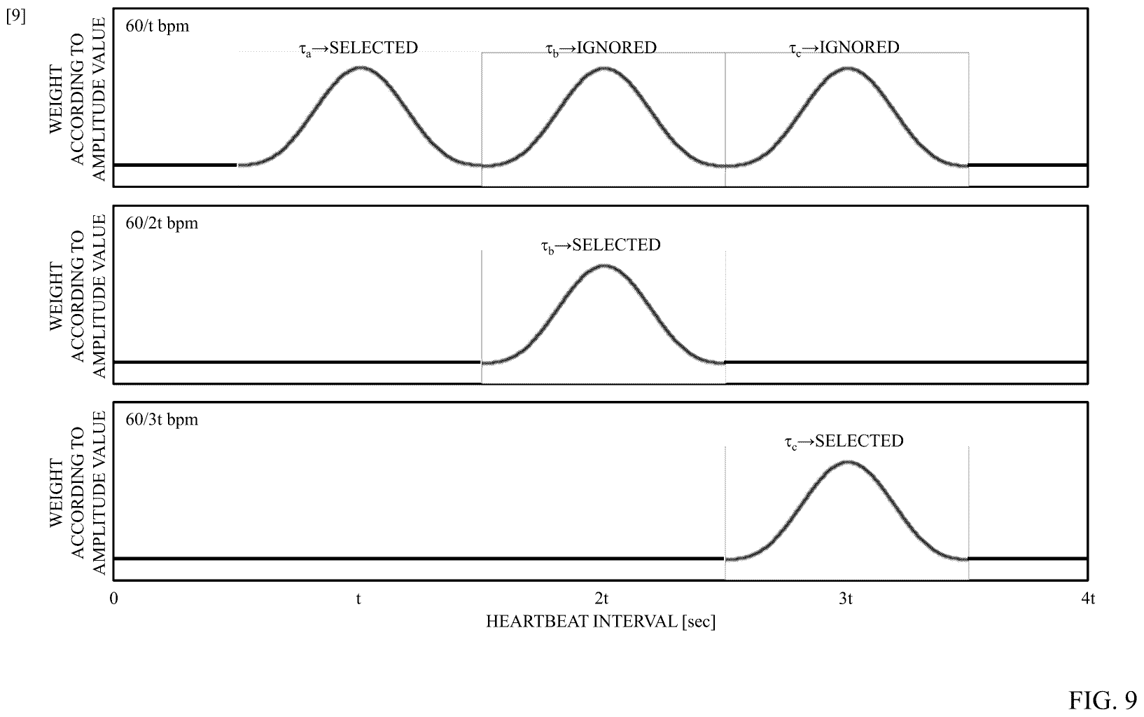

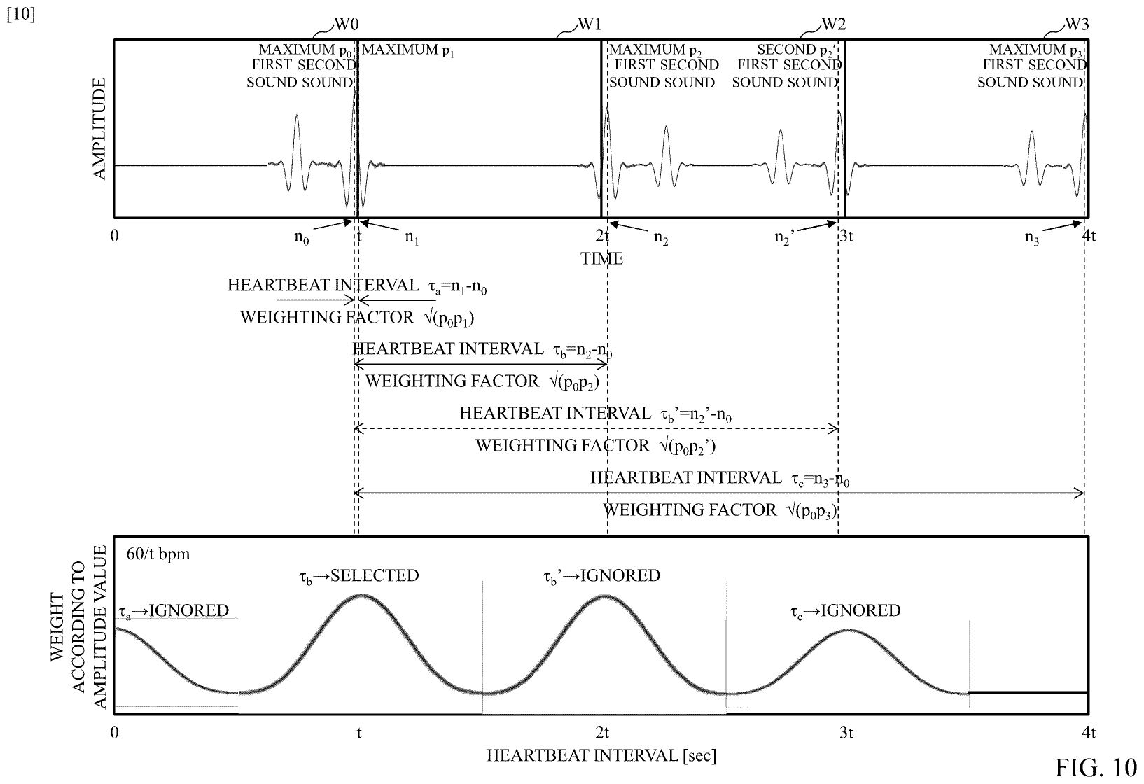

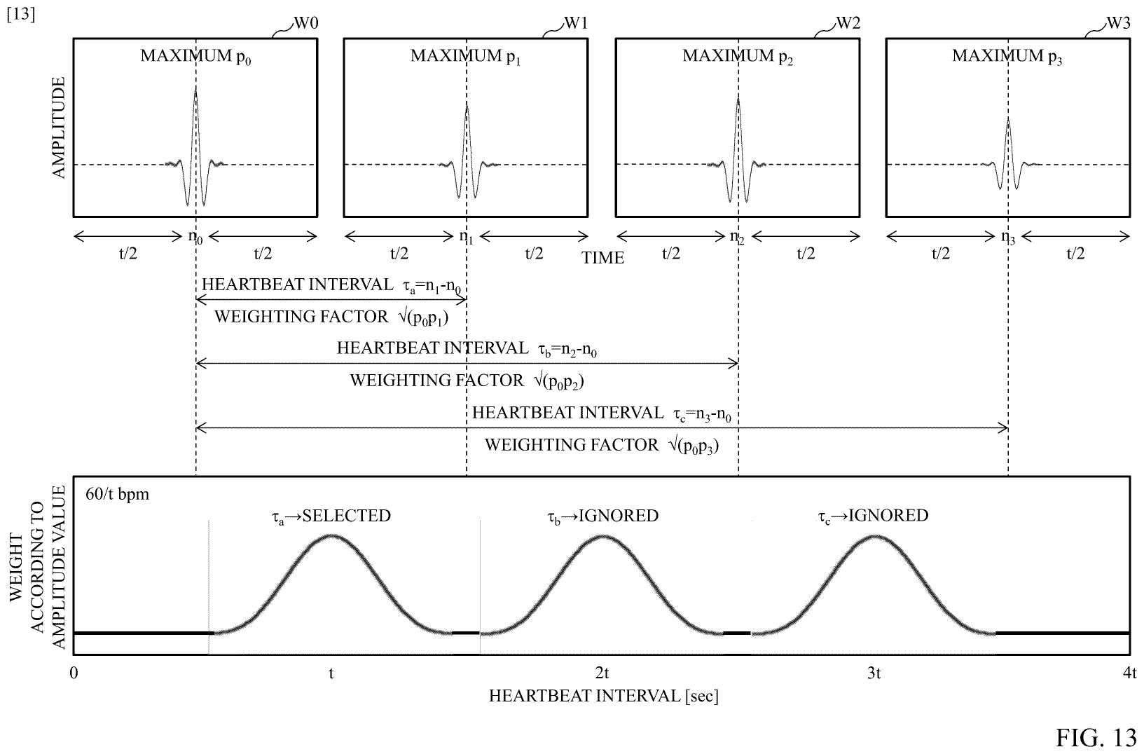

In the heartbeat observation time window Wb, the first sound and the second sound in one heartbeat are included in an out-of-phase state (the phase relationship varies depending on individual differences or animal species). Then, in the spectrogram S, after the first sound and the second sound in one heartbeat are synthesized in a mutually destructive state, the frequency components (ri[n], rq[n]) and (li[n], lq[n]) are extracted. Alternatively, in the band-pass filter result B, after the first sound and the second sound in one heartbeat are synthesized in a mutually destructive state, the frequency component b[n] is extracted. However, if τₐ, τ_{b}, τ_{c}, and the like are considered as illustrated in FIG. 9, FIG. 10, and FIG. 13, the avoidance of multiple times counting can be approximately completely ensured.

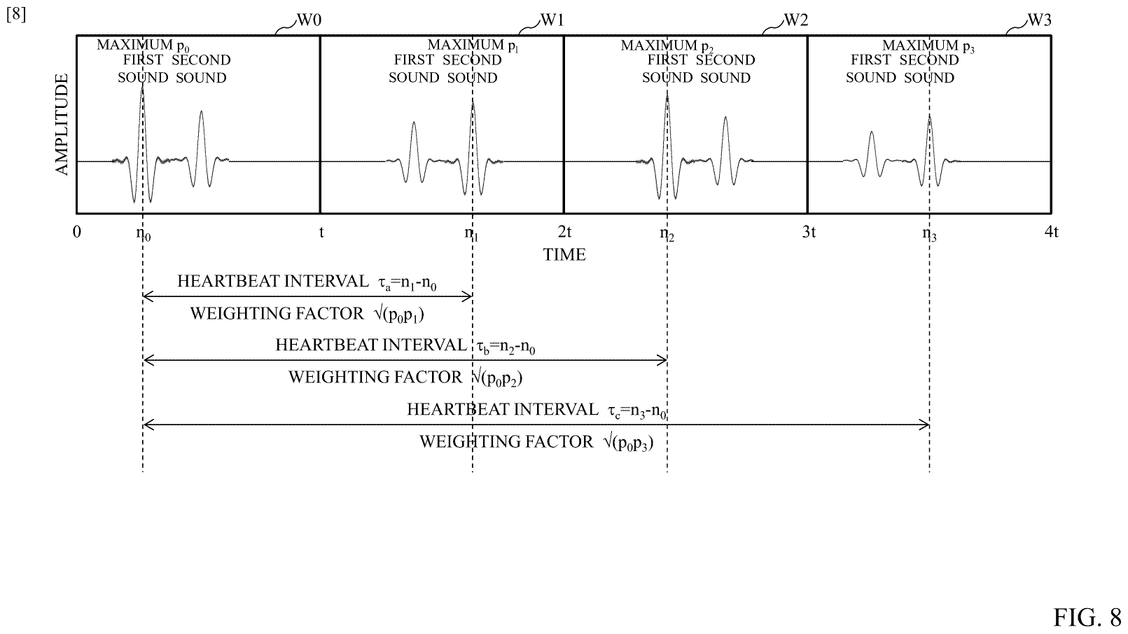

In the heartbeat observation time window Wc, only the first sound in one heartbeat is included (the number of micro vibrations varies depending on the situation of heartbeat and respiration). Then, in the spectrogram S, only for the first sound in one heartbeat, the frequency components (ri[n], rq[n]) and (li[n], lq[n]) are extracted. Alternatively, in the band-pass filter result B, only for the first sound in one heartbeat, the frequency component b[n] is extracted. However, if the peak detection time window is applied as illustrated in FIG. 8 to FIG. 13, the avoidance of multiple times counting can be approximately completely ensured.

In the heartbeat observation time window Wd, only the second sound in one heartbeat is included (the number of micro vibrations varies depending on the situation of heartbeat and respiration). Then, in the spectrogram S, only for the second sound in one heartbeat, the frequency components (ri[n], rq[n]) and (li[n], lq[n]) are extracted. Alternatively, in the band-pass filter result B, only for the second sound in one heartbeat, the frequency component b[n] is extracted. However, if the peak detection time window is applied as illustrated in FIG. 8 to FIG. 13, the avoidance of multiple times counting can be approximately completely ensured.

(Procedure of Heartbeat Detection Processing of this Disclosure: First Heartbeat Detection Processing)

FIG. 8 to FIG. 10 illustrate specific examples of first heartbeat detection processing of this disclosure. The heartbeat peak extraction unit 3 extracts maximum amplitude peaks of the amplitude peaks from the frequency components (ri[n], rq[n]), (li[n], lq[n]), b[n], or (mi[n], mq[n]) within predefined peak detection time windows W0, W1, W2, and W3 (Step S2).

In FIG. 8, the amplitude peaks of the first sound and the second sound in one heartbeat are present within the peak detection time windows W0, W1, W2, and W3 with a predefined time width t (a peak detection time window is slightly longer than a heartbeat observation time window). Within the peak detection time window W0, the amplitude peak of the first sound has the peak time no and a maximum amplitude value p₀. Within the peak detection time window W1, the amplitude peak of the second sound has the peak time n₁ and a maximum amplitude value p₁. Within the peak detection time window W2, the amplitude peak of the first sound has the peak time n₂ and a maximum amplitude value p₂. Within the peak detection time window W3, the amplitude peak of the second sound has the peak time n₃ and a maximum amplitude value p₃.

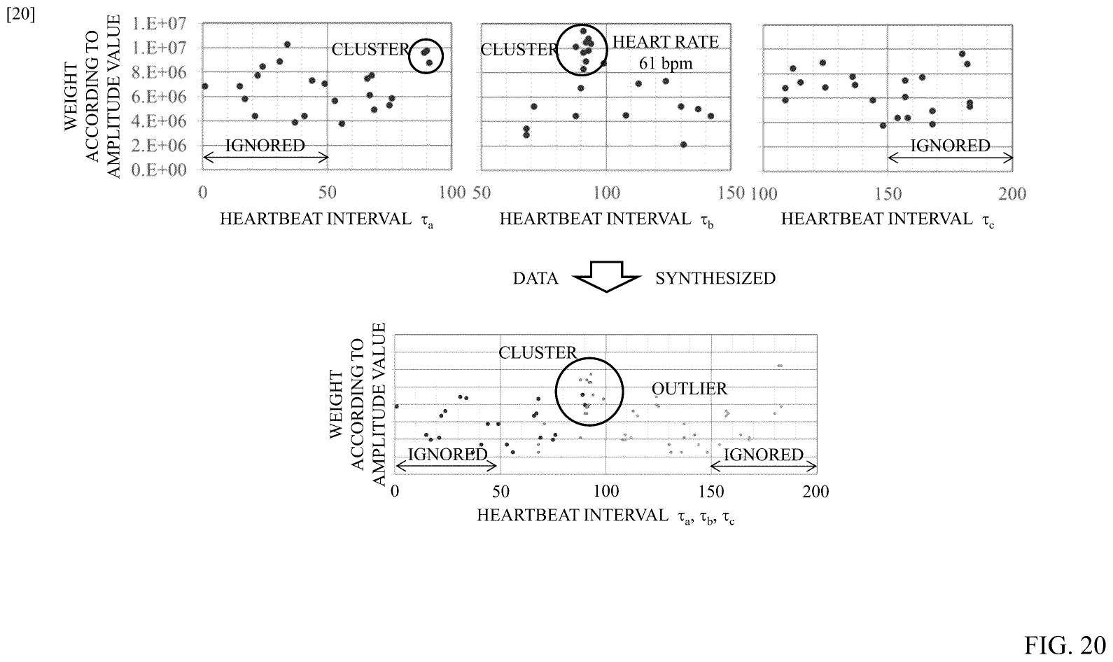

The heart rate calculation unit 4 calculates an average heart rate with increasing a weight of the time interval of the amplitude peaks as amplitude values of the amplitude peaks increase (Step S3). Alternatively, the heart rate calculation unit 4 calculates the heart rate based on clustering of two-dimensional data constituted of the time interval of the amplitude peaks and the weight of the time interval with increasing the weight of the time interval of the amplitude peaks as the amplitude values of the amplitude peaks increase (Step S3, FIG. 20).

The heart rate calculation unit 4 calculates the heart rate based on the time interval of the maximum amplitude peaks extracted in adjacent or non-adjacent predefined peak detection time windows (Step S3). In FIG. 8, between the peak detection time windows W0 and W1, a heartbeat interval τₐ is n₁−n₀ and has a weighting factor of √(p₀p₁). Between the peak detection time windows W0 and W2, a heartbeat interval τ_{b} is n₂−n₀ and has a weighting factor of √(p₀p₂). Between the peak detection time windows W0 and W3, a heartbeat interval τ_{c} is n₃−n₀ and has a weighting factor of √(p₀p₃).

In FIG. 9, average heartbeat intervals τ_{a,ave}, τ_{b,ave}, and τ_{c,ave} are calculated as Math. 1 to Math. 3. Here, when a peak detection time window Wm (m=0 to M−1) is used as a reference, τₐ=τₐ[m], τ_{b}=τ_{b}[m], τ_{c}=τ_{c}[m], p₀=p₀[m], p₁=p₁[m], p₂=p₂[m], and p₃=p₃[m]. Then, the average heartbeat intervals τ_{a,ave}, τ_{b,ave}, and τ_{c,ave} are average values from a start (m=0) to an end (m=M−1) of a detection period of the heart rate.

$$\overline{{τ}_{a}} = \frac{{\sum }_{m = 0}^{M - 1} {τ}_{a} [ m ] \cdot \sqrt{{p}_{1} [ m ] \cdot {p}_{0} [ m ]}}{{\sum }_{m = 0}^{M - 1} \sqrt{{p}_{1} [ m ] \cdot {p}_{0} [ m ]}} \qquad [ Math . 1 ]$$

$$\overline{{τ}_{b}} = \frac{{\sum }_{m = 0}^{M - 1} {τ}_{b} [ m ] \cdot \sqrt{{p}_{2} [ m ] \cdot {p}_{0} [ m ]}}{{\sum }_{m = 0}^{M - 1} \sqrt{{p}_{2} [ m ] \cdot {p}_{0} [ m ]}} \qquad [ Math . 2 ]$$

$$\overline{{τ}_{c}} = \frac{{\sum }_{m = 0}^{M - 1} {τ}_{c} [ m ] \cdot \sqrt{{p}_{3} [ m ] \cdot {p}_{0} [ m ]}}{{\sum }_{m = 0}^{M - 1} \sqrt{{p}_{3} [ m ] \cdot {p}_{0} [ m ]}} \qquad [ Math . 3 ]$$

When the average heart rate is 60/3t (bpm), a weight of the heartbeat interval τ_{c} has a peak in the average heartbeat interval τ_{c,ave}, and the average heartbeat interval τ_{c,ave} is selected. When the average heart rate is 60/2t (bpm), a weight of the heartbeat interval τ_{b} has a peak in the average heartbeat interval τ_{b,ave}, and the average heartbeat interval τ_{b,ave} is selected. When the average heart rate is 60/t (bpm), weights of the heartbeat intervals τₐ, τ_{b}, and τ_{c} have peaks in the average heartbeat intervals τ_{a,ave}, τ_{b,ave}, and τ_{c,ave}, and only the average heartbeat interval τ_{a,ave} is selected. As illustrated in the lower stage of FIG. 20 described below, synthetic two-dimensional data may be created.

In FIG. 10, the amplitude peaks of the first sound and the second sound in one heartbeat are present at boundaries of the peak detection time windows W0, W1, W2, and W3 with the predefined time width t (a peak detection time window is slightly longer than a heartbeat observation time window). Within the peak detection time window W0, the amplitude peak of the second sound has the peak time no and the maximum amplitude value p₀. Within the peak detection time window W1, the amplitude peak of the second sound has the peak time n₁ (=t) and the maximum amplitude value p₁. Within the peak detection time window W2, the amplitude peak of the first sound for the first time has the peak time n₂ and the maximum amplitude value p₂, and the amplitude peak of the second sound for the second time has the peak time n₂′ and a second amplitude value p₂′. Within the peak detection time window W3, the amplitude peak of the second sound has the peak time n₃ and the maximum amplitude value p₃.

In FIG. 10, between the peak detection time windows W0 and W1, the heartbeat interval τₐ is n₁−n₀ and has a weighting factor of √(p₀p₁) (since the heartbeat interval τₐ is a short time, it is ignored as described below). Between the peak detection time windows W0 and W2, the heartbeat interval τ_{b} is n₂−n₀ and has a weighting factor of √(p₀p₂), and a heartbeat interval τ_{b}′ is n₂′−n₀ and has a weighting factor of √(p₀p₂′). Between the peak detection time windows W0 and W3, the heartbeat interval τ_{c} is n₃−n₀ and has a weighting factor of √(p₀p₃).

In FIG. 10, average heartbeat intervals τ_{a,ave}=Στₐ√(p₀p₁)/Σ√(p₀p₁), τ_{b,ave}=Στ_{b}(p₀p₂)/Σ√(p₀p₂), τ_{b,ave}′=Στ_{b}′√(p₀p₂′)/Σ√(p₀p₂′), and τ_{c,ave}=Στ_{c}√(p₀p₃)/Σ√(p₀p₃) are calculated (the sums are obtained over the detection period of the heart rate). When the average heart rate is 60/t (bpm), weights of the heartbeat intervals τₐ, τ_{b}, τ_{b}′, and τ_{c} have peaks in the average heartbeat intervals τ_{a,ave}, τ_{b,ave}, τ_{b,ave}′, and τ_{c,ave}, and only the average heartbeat interval τ_{b,ave} is selected. As illustrated in the lower stage of FIG. 20 described below, synthetic two-dimensional data may be created.

Thus, even when only one characteristic micro vibration in one heartbeat is included in a predefined heartbeat observation time window, only the maximum amplitude peak is extracted in a predefined peak detection time window, and therefore, the avoidance of multiple times counting can be approximately completely ensured. However, depending on a time width of the predefined peak detection time window, an upper limit 60/t (bpm) and a lower limit 60/3t (bpm) of the heart rate are narrowed to some extent. Then, since the time interval of the heartbeat amplitude peaks is weighted according to the magnitudes of the amplitude values of the heartbeat amplitude peaks, the calculation of the heart rate can be performed with high accuracy. Furthermore, since the weight of the time interval of the heartbeat amplitude peaks is calculated according to the magnitudes of the amplitude values of the heartbeat amplitude peaks, the calculation of the heart rate can be performed with high accuracy.

(Procedure of Heartbeat Detection Processing of this Disclosure: Second Heartbeat Detection Processing)

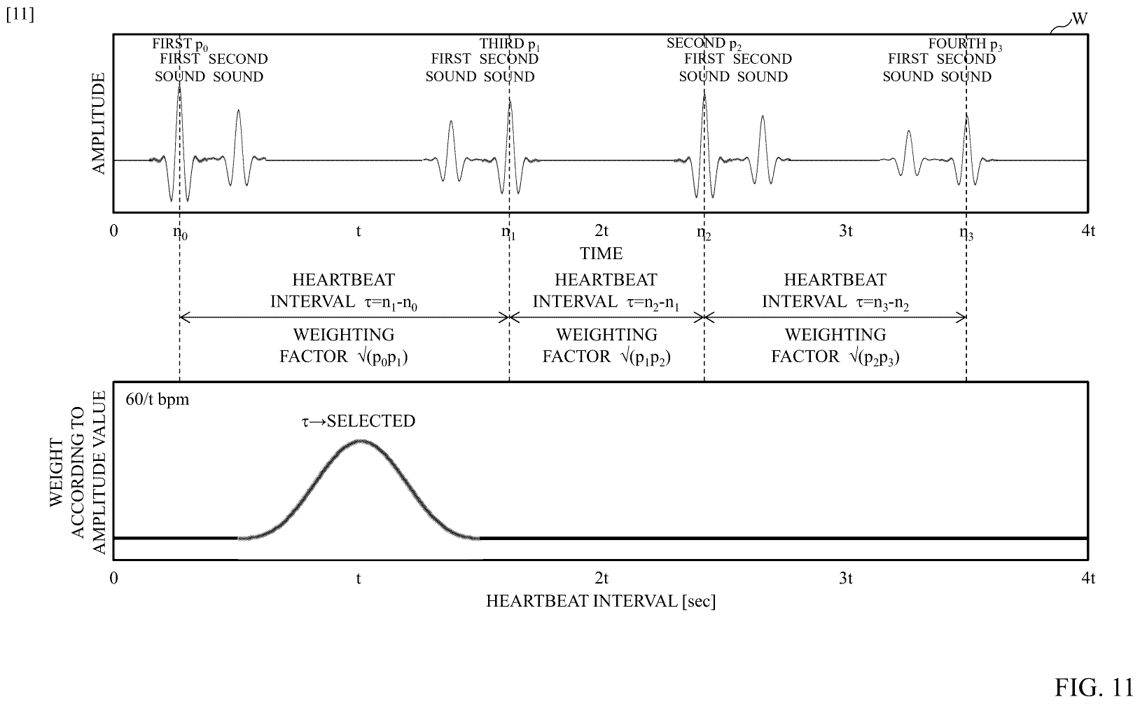

FIG. 11 illustrates a specific example of second heartbeat detection processing of this disclosure. The heartbeat peak extraction unit 3 extracts amplitude peaks within a predefined number or with a predefined amplitude or more from the frequency components (ri[n], rq[n]), (li[n], lq[n]), b[n], or (mi[n], mq[n]) in a predefined peak detection time window W, which is longer than the predefined peak detection time windows W0 to W3 (Step S2).

In FIG. 11, the amplitude peaks of the first sound and the second sound in four heartbeats are present in the peak detection time window W (the time width is 4t). In chronological order in the peak detection time window W (the time width is 4t), the amplitude peak of the first sound has the peak time no and a first amplitude value p₀, the amplitude peak of the second sound has the peak time n₁ and a third amplitude value p₁, the amplitude peak of the first sound has the peak time n₂ and a second amplitude value p₂, and the amplitude peak of the second sound has the peak time n₃ and a fourth amplitude value p₃. A known QRS detection algorithm, such as Engelse and Zeelenberg, may be used to extract the amplitude peaks within a predefined number or with a predefined amplitude or more.

The heart rate calculation unit 4 calculates an average heart rate with increasing a weight of the time interval of the amplitude peaks as the amplitude values of the amplitude peaks increase (Step S3). Alternatively, the heart rate calculation unit 4 calculates the heart rate based on clustering of two-dimensional data constituted of the time interval of the amplitude peaks and the weight of the time interval with increasing the weight of the time interval of the amplitude peaks as the amplitude values of the amplitude peaks increase (Step S3, FIG. 20).

In FIG. 11, in chronological order in the peak detection time window W (the time width is 4t), the first adjacent heartbeat interval r is n₁−n₀ and has a weighting factor of √(p₀p₁), the second adjacent heartbeat interval τ is n₂−n₁ and has a weighting factor of √(p₁p₂), and the third adjacent heartbeat interval τ is n₃−n₂ and has a weighting factor of √(p₂p₃).

In FIG. 11, an average heartbeat interval τₐᵥₑ=Στ√(pₙpₙ₊₁)/Σ√(pₙpₙ₊₁) is calculated (the sum is obtained over the detection period of the heart rate). When the average heart rate is 60/t (bpm), the weight of the heartbeat interval τ has a peak in the average heartbeat interval τₐᵥₑ. As illustrated in the lower stage of FIG. 20 described below, synthetic two-dimensional data may be created.

Thus, even when only one characteristic micro vibration in one heartbeat is included in a predefined heartbeat observation time window, amplitude peaks within a predefined number or with a predefined amplitude or more are extracted in a predefined peak detection time window, and therefore, an upper limit and a lower limit of the heart rate can be extended to a sufficient degree compared with FIG. 8 to FIG. 10. However, depending on the predefined number and the predefined amplitude, the occurrence of multiple times counting is brought about to some extent. Then, since the time interval of the heartbeat amplitude peaks is weighted according to the magnitudes of the amplitude values of the heartbeat amplitude peaks, the calculation of the heart rate can be performed with high accuracy. Furthermore, since the weight of the time interval of the heartbeat amplitude peaks is calculated according to the magnitudes of the amplitude values of the heartbeat amplitude peaks, the calculation of the heart rate can be performed with high accuracy.

### (Procedure of Heartbeat Detection Processing of This Disclosure: Third Heartbeat Detection Processing)

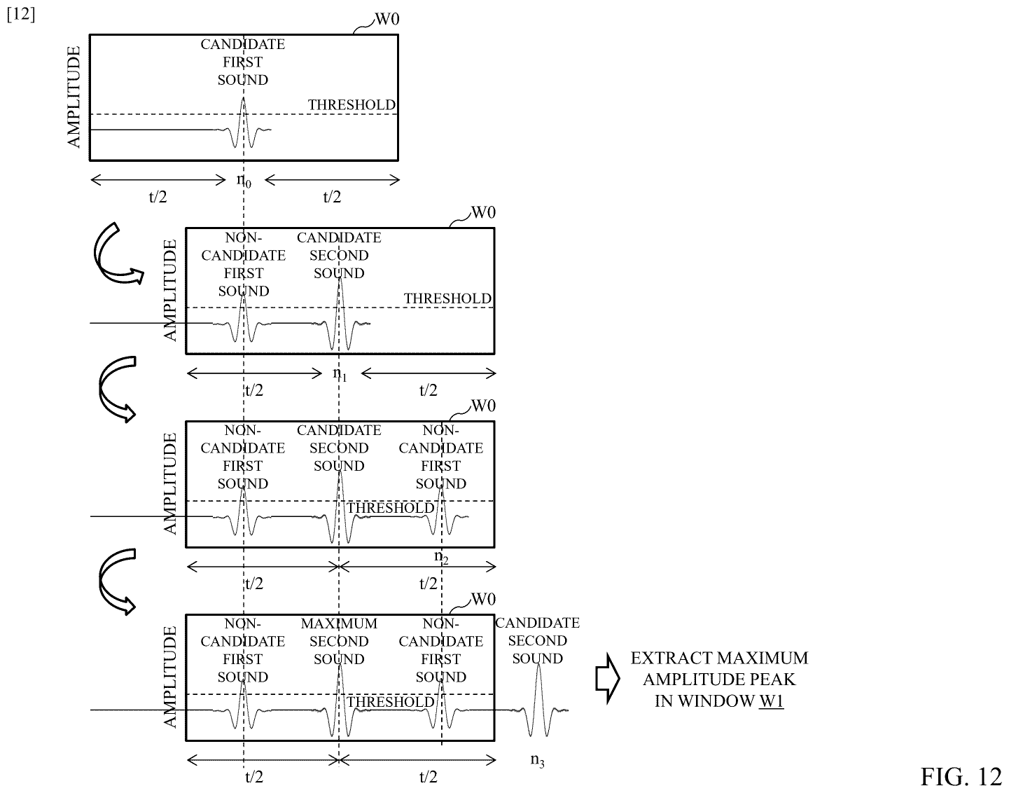

FIG. 12 and FIG. 13 illustrate specific examples of third heartbeat detection processing of this disclosure. The heartbeat peak extraction unit 3 moves the predefined peak detection time windows W0, W1, W2, and W3 so as to extract the maximum amplitude peaks in time domains excluding vicinities of both ends of the predefined peak detection time windows W0, W1, W2, and W3 (Step S2). This is for not causing the short-time heartbeat interval τₐ of FIG. 10.

In FIG. 12, the peak detection time window W0 with the predefined time width t is moved according to the amplitude peaks of the first sound and the second sound in one heartbeat (a peak detection time window is slightly longer than a heartbeat observation time window). In the first stage of FIG. 12, an amplitude peak of a first sound has an amplitude value greater than a predefined noise threshold and is selected as a candidate for the maximum amplitude peak. Then, the peak detection time window W0 is set such that time no of the candidate for the maximum amplitude peak is located at the central time of the peak detection time window W0.

In the second stage of FIG. 12, an amplitude peak of a second sound has an amplitude value greater than the predefined noise threshold, has the amplitude value greater than the previous candidate for the maximum amplitude peak, and is selected as a new candidate for the maximum amplitude peak. Then, the peak detection time window W0 is moved by an amount of time n₁−n₀ such that time n₁ of the new candidate for the maximum amplitude peak is located at the central time of the peak detection time window W0.

In the third stage of FIG. 12, an amplitude peak of a first sound has an amplitude value greater than the predefined noise threshold, has the amplitude values smaller than the previous candidate for the maximum amplitude peak, and is not selected as a new candidate for the maximum amplitude peak. Then, the peak detection time window W0 is fixed with no change such that the time n₁ of the previous candidate for the maximum amplitude peak is located at the central time of the peak detection time window W0.

In the fourth stage of FIG. 12, an amplitude peak of a second sound has an amplitude value greater than the predefined noise threshold but is not included in the previous peak detection time window W0. Therefore, the new peak detection time window W1 is moved according to the amplitude peaks of the first sound and the second sound in one heartbeat. That is, the amplitude peak of the second sound has an amplitude value greater than the predefined noise threshold and is selected as a candidate for the maximum amplitude peak. Then, the peak detection time window W1 is set such that time n₃ of the candidate for the maximum amplitude peak is located at the central time of the peak detection time window W1. Subsequently, similar processing is repeated.

The heart rate calculation unit 4 calculates an average heart rate with increasing a weight of the time interval of the amplitude peaks as the amplitude values of the amplitude peaks increase (Step S3). Alternatively, the heart rate calculation unit 4 calculates the heart rate based on clustering of two-dimensional data constituted of the time interval of the amplitude peaks and the weight of the time interval with increasing the weight of the time interval of the amplitude peaks as the amplitude values of the amplitude peaks increase (Step S3, FIG. 20).

The heart rate calculation unit 4 calculates the heart rate based on the time interval of the maximum amplitude peaks extracted in adjacent or non-adjacent predefined peak detection time windows (Step S3). In FIG. 13, between the peak detection time windows W0 and W1, the heartbeat interval τₐ is n₁−n₀ and has a weighting factor of √(p₀p₁). Between the peak detection time windows W0 and W2, the heartbeat interval τ_{b} is n₂−n₀ and has a weighting factor of √(p₀p₂). Between the peak detection time windows W0 and W3, the heartbeat interval τ_{c} is n₃−n₀ and has a weighting factor of √(p₀p₃).

In FIG. 13, the average heartbeat intervals τ_{a,ave}=Στₐ√(p₀p₁)/Σ√(p₀p₁), τ_{b,ave}=Στ_{b}√(p₀p₂)/Σ√(p₀p₂), and τ_{c,ave}=Στ_{c}√(p₀p₃)/Σ√(p₀p₃) are calculated (the sums are obtained over the detection period of the heart rate). When the average heart rate is 60/t (bpm), the weights of the heartbeat intervals τₐ, τ_{b}, and τ_{c} have peaks in the average heartbeat intervals τ_{a,ave}, τ_{b,ave}, and τ_{c,ave}, and only the average heartbeat interval τ_{a,ave} is selected. Alternatively, as illustrated in the lower stage of FIG. 20 described below, synthetic two-dimensional data may be created.

Thus, since one characteristic micro vibration in one heartbeat is made to avoid straddling an adjacent predefined peak detection time window, and therefore, the upper limit of the heart rate can be extended without causing the short-time heartbeat interval τₐ of FIG. 10. Meanwhile, when one characteristic micro vibration in one heartbeat is smaller than the predefined noise threshold, it is not selected as a candidate for the maximum amplitude peak, and therefore, the lower limit of the heart rate can be extended. Then, since the time interval of the heartbeat amplitude peaks is weighted according to the magnitudes of the amplitude values of the heartbeat amplitude peaks, the calculation of the heart rate can be performed with high accuracy. Furthermore, since the weight of the time interval of the heartbeat amplitude peaks is calculated according to the magnitudes of the amplitude values of the heartbeat amplitude peaks, the calculation of the heart rate can be performed with high accuracy.

In the first to third heartbeat detection processing, even when τₐᵥₑ=Στ√(pₙpₙ₊₁)/Σ√(pₙpₙ₊₁) is calculated as an average heartbeat interval, it is not necessary to prepare all class values of T. Then, after the weight of the time interval of the amplitude peaks is calculated, a weighted average of the two-dimensional data constituted of the time interval of the amplitude peaks and the weight of the time interval is calculated. Accordingly, the detection period of the heart rate can be shortened. In addition, when τₙ←(1−λ)τₙ+λτₙ₋₁ is calculated as an average heartbeat interval, it is only necessary to set a forgetting coefficient X according to √(pₙ₋₁pₙ), and compared with a case when the weighted average of the two-dimensional data in the detection period of the heart rate is calculated, burdens of calculating τₙ can be alleviated. Note that as the weighting factor, in addition to setting √(pₙ₋₁pₙ), max(pₙ₋₁, pₙ) may be set, or min(pₙ₋₁, pₙ) may be set.

Here, when τₙ←(1−λ)τₙ+λτₙ₋₁ is calculated as an average heartbeat interval, making the forgetting coefficient λ small (large) makes it easy (hard) to remove noise but hard (easy) to shorten time for convergence. Then, when a first-order IIR filter is applied, the filter can be simplified compared with a case when a moving average filter and an FIR filter are applied.

In this embodiment, a heart rate of 60/r (bpm) is calculated with high accuracy based on the time interval T (seconds) of the amplitude peaks. As a modification, a heart rate of 60 k/To (bpm) may be calculated more simply based on a count k (pieces) of the amplitude peaks extracted within a predetermined time To (seconds). For example, a heart rate of 60 bpm can be calculated by extracting only 10 amplitude peaks in a measurement for 10 seconds. However, if one more amplitude peak is erroneously extracted in the measurement for 10 seconds, a heart rate of 66 bpm is calculated, and an error of +10% occurs. Accordingly, the accuracy is higher when the time interval of the amplitude peaks is used, and a calculation amount is fewer when the count of the amplitude peaks extracted is used.

(Procedure of Respiration Detection Processing of this Disclosure)

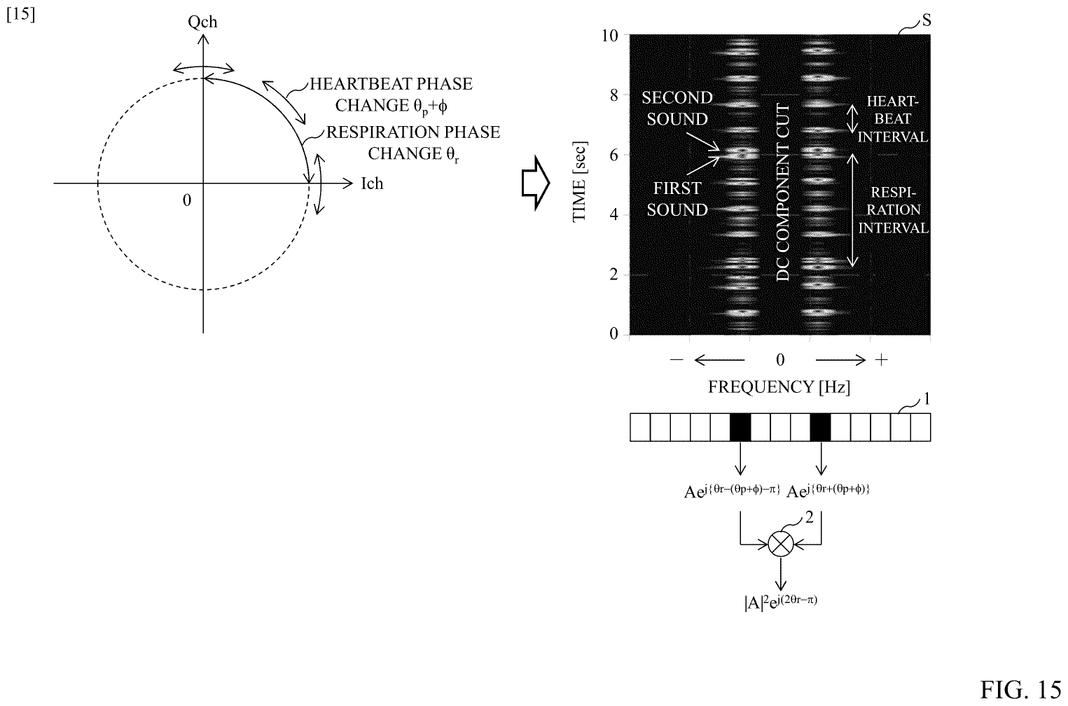

FIG. 14 illustrates a procedure of respiration detection processing of this disclosure. The heartbeat component extraction unit 1 extracts the frequency components (ri[n], rq[n]) and (li[n], lq[n]), and the heartbeat component multiplication unit 2 calculates the frequency component (mi[n], mq[n]) (Step S4). The respiration phase extraction unit 5 extracts a respiration phase change caused by the micro vibration of the respiration, with a heartbeat phase change caused by the micro vibration of the heartbeat removed, from the frequency component (mi[n], mq[n]) (Step S5). The respiration rate calculation unit 6 calculates the respiration rate based on a frequency component (for example, a Fourier transform component) of the respiration phase change (Step S6). Specifically, the processing from FIG. 15 to FIG. 18 is performed.

FIG. 15 to FIG. 17 illustrate a principle of the respiration detection processing of this disclosure. In FIG. 15, on the radar signal or the ultrasound signal (carrier wave band) reflecting off the body surface of the patient P, heartbeat phase modulation caused by the micro vibration of the heartbeat is performed, and respiration phase modulation caused by the micro vibration of the respiration is also performed. Here, on the radar signal or the ultrasound signal (carrier wave band) reflecting off the body surface of the patient P, although heartbeat amplitude modulation is also performed, the heartbeat amplitude modulation is uncertain compared with the heartbeat phase modulation and therefore disadvantageous for highly accurate respiration extraction, and although respiration amplitude modulation is also performed, the respiration amplitude modulation is uncertain compared with the respiration phase modulation and therefore disadvantageous for highly accurate respiration extraction.

Then, the heartbeat component extraction unit 1 extracts Ae^{i{θr+(θP+ϕ)}} (A is an amplitude, θᵣ is a respiration phase change, θₚ is a heartbeat phase change, and ϕ is an initial phase of the heartbeat phase change) as a positive frequency component caused by the micro vibration of the heartbeat and extracts Ae^{j{θr-(θP+ϕ)-π}} (π is a phase difference between upper and lower sideband waves of phase modulation) as a negative frequency component caused by the micro vibration of the heartbeat. Then, the heartbeat component multiplication unit 2 calculates Ae^{j{θr+(θp+ϕ)}}*Ae^{j{θr-(θp+ϕ)-π}}=|A|²e^{j(2θr-π)} as complex multiplication of the positive and negative frequency components. Accordingly, from complex multiplication of the positive and negative frequency components |A|²e^{j(2θr-π)}, the respiration phase extraction unit 5 can extract the respiration phase change θᵣ caused by the micro vibration of the respiration, with the heartbeat phase change θₚ+ϕ caused by the micro vibration of the heartbeat removed.

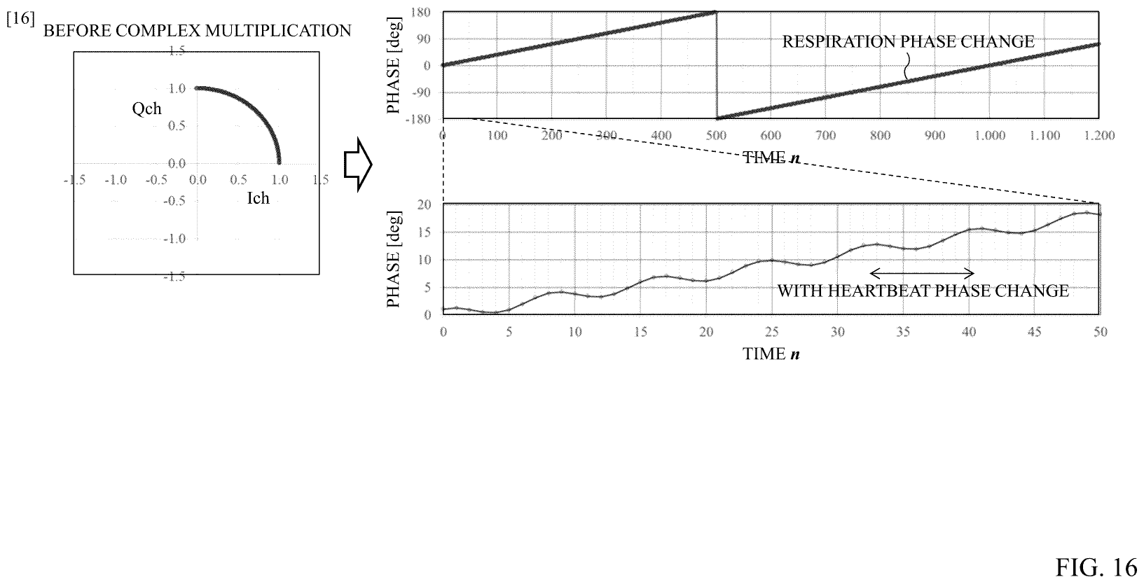

FIG. 16 illustrates a simulation result at a stage before the heartbeat component multiplication unit 2 calculates |A|²e^{j(2θr-π)} as complex multiplication of the positive and negative frequency components. In the left section of FIG. 16, while the respiration phase change θᵣ changes from 0 to π/2, the heartbeat phase change θₚ+ϕ superimposes minute changes. In the upper right section of FIG. 16, a state where the respiration phase change θᵣ changes is mainly observed, and in the lower right section of FIG. 16 (a part of the time domain is enlarged), a state where the respiration phase change θᵣ changes is observed, and a state where the heartbeat phase change θₚ+ϕ changes is also largely observed.

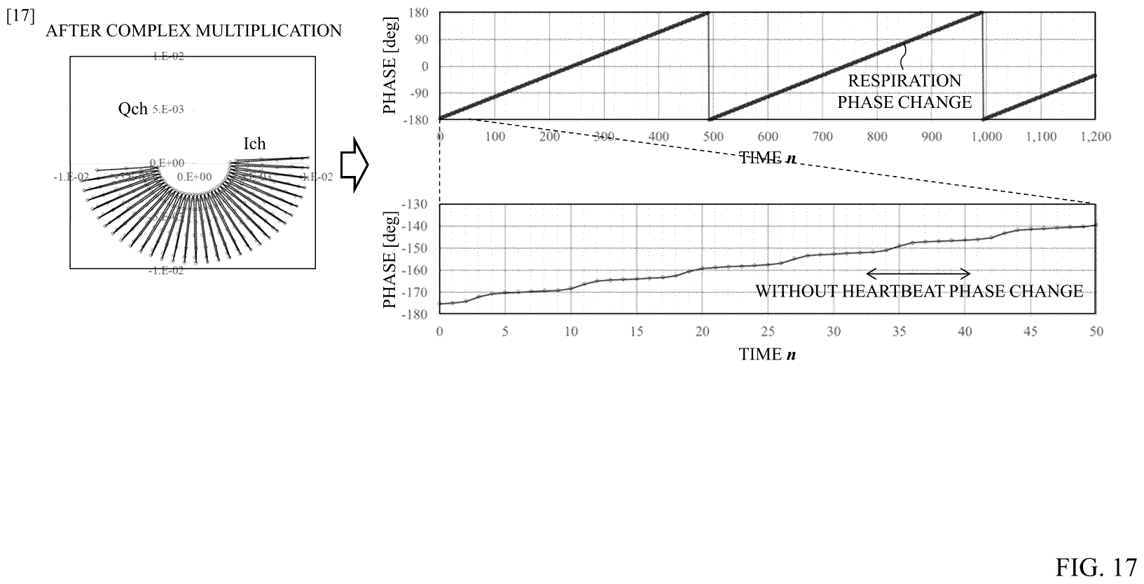

FIG. 17 illustrates a simulation result at a stage after the heartbeat component multiplication unit 2 calculates |A|²e^{j(2θr-π)} as complex multiplication of the positive and negative frequency components. In the left section of FIG. 17, while a respiration phase change 2θᵣ−π changes from −π to 0, the heartbeat phase change θₚ+ϕ does not superimpose minute changes (although a change in amplitude is observed, a change in phase is a monotonous change). In the upper right section of FIG. 17, a state where the respiration phase change 2θᵣ−π changes is mainly observed, and in the lower right section of FIG. 17 (a part of the time domain is enlarged), although a state where the respiration phase change 2θᵣ−π changes is observed, a state where the heartbeat phase change θₚ+ϕ changes is hardly observed.

Thus, since the frequency component caused by the micro vibration of the heartbeat is extracted, the improvement of robustness and the reduction of the effects of disturbances can be ensured. Then, since the respiration rate is calculated based on the frequency component of the respiration phase change with the heartbeat phase change removed, the avoidance of multiple times counting and the extension of a detection range can be ensured in consideration of a reflected signal from a chest without considering a reflected signal from an abdomen.

Furthermore, since the data that serves as a basis for calculating the respiration rate is different from the data that serves as a basis for calculating the heart rate (described above), the separation of respiration and heartbeat can be ensured.

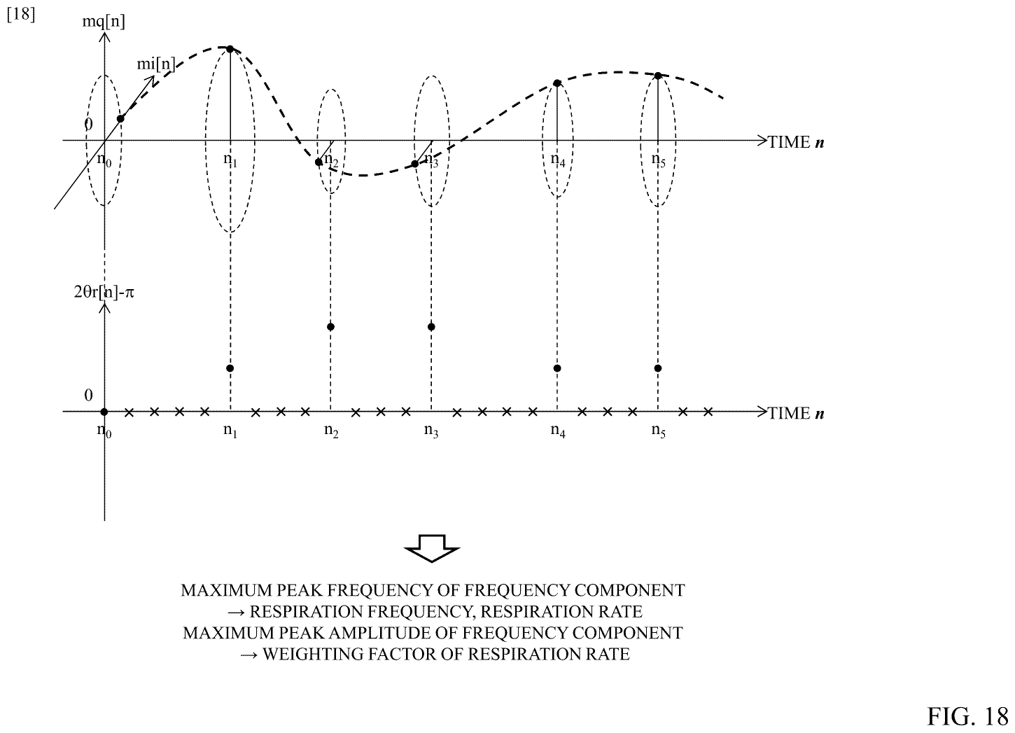

FIG. 18 illustrates a specific example of the respiration detection processing of this disclosure. The respiration phase extraction unit 5 extracts amplitude peaks caused by the micro vibration of the heartbeat from complex multiplication of the positive and negative frequency components |A|²e^{j(2θr-π)} and extracts the respiration phase change 2θᵣ−π at the amplitude peaks while applying zero padding between the amplitude peaks without extracting the respiration phase change 2θᵣ−π (Step S5). The respiration rate calculation unit 6 calculates an average respiration rate with increasing a weight of the respiration rate as a maximum peak amplitude of the frequency component of the respiration phase change 2θᵣ−π increases (Step S6).

In FIG. 18, at the peak time no, an amplitude peak is present, and the respiration phase change 2θᵣ−π=0 is extracted. At the peak time n₁, an amplitude peak is present, and the respiration phase change 2θᵣ−π=π/2 is extracted. At the peak time n₂, an amplitude peak is present, and the respiration phase change 2θᵣ−π=π is extracted. At the peak time n₃, an amplitude peak is present, and the respiration phase change 2θᵣ−π=π is extracted. At the peak time n₄, an amplitude peak is present, and the respiration phase change 2θᵣ−π=π/2 is extracted. At the peak time n₅, an amplitude peak is present, and the respiration phase change 2θᵣ−π=π/2 is extracted. Between the peak times n₀, n₁, n₂, n₃, n₄, and n₅, amplitude peaks are not present, and I=Q=0 is applied as zero padding.

In FIG. 18, based on a maximum peak frequency of the frequency component of the respiration phase change 2θᵣ−π, the respiration frequency and therefore the respiration rate are calculated. Then, based on the maximum peak amplitude of the frequency component of the respiration phase change 2θᵣ−π, the weighting factor of the respiration rate is calculated. Here, similarly to a case when the heart rate is calculated, even when the respiration rate is calculated, the weight of the respiration rate according to the maximum peak amplitude may be calculated, and the forgetting coefficient λ may be set according to the weighting factor.

Thus, in consideration of the fluctuation of the heartbeat, information on the respiration phase change is used at heartbeat amplitude peak time while the information on the respiration phase change is not used between the heartbeat amplitude peaks, and therefore, the calculation of the respiration rate can be performed with high accuracy. Here, instead of performing a frequency conversion of the respiration phase change θᵣ, a frequency conversion of the respiration phase change 2θᵣ−π is performed (2θᵣ is doubled compared with θᵣ), and therefore, the maximum peak frequency of the frequency component can be calculated with high accuracy. Then, since the respiration rate is weighted according to the magnitude of the maximum peak amplitude of the frequency component of the respiration phase change, the calculation of the respiration rate can be performed with high accuracy.

In this embodiment, the respiration phase change 2θᵣ−π caused by the micro vibration of the respiration is extracted from the frequency component (mi[n], mq[n]). Simultaneously with this, a respiration amplitude change |A|² caused by the micro vibration of the respiration is also extracted from the frequency component (mi[n], mq[n]). As a reason for this, complex multiplication of the positive and negative frequency components |A|²e^{j(2θr-π)} are calculated.

As a modification, one of the respiration phase change and the respiration amplitude change caused by the micro vibration of the respiration may be extracted from the frequency component (mi[n], mq[n]). As another example, one or both of the respiration phase change and the respiration amplitude change caused by the micro vibration of the respiration may be extracted from the frequency component (ri[n], rq[n]), (li[n], lq[n]), or b[n].

(Result of Heartbeat and Respiration Detection Processing of this Disclosure)

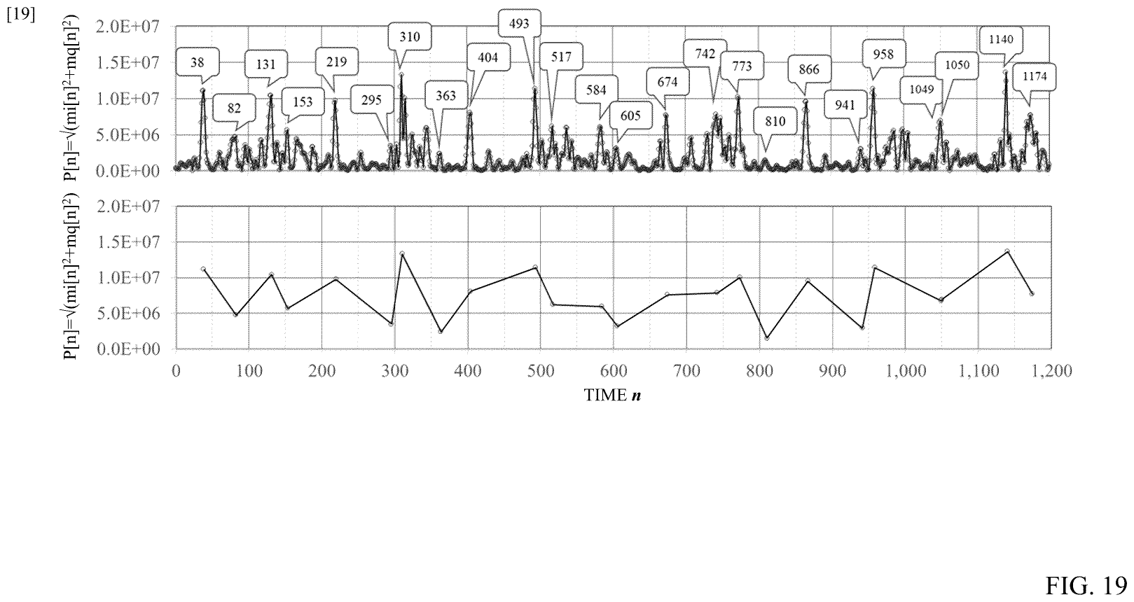

FIG. 19 and FIG. 20 illustrate results of the heartbeat detection processing of this disclosure. The upper stage of FIG. 19 illustrates a temporal change of an amplitude P[n]=√(mi[n]²+mq[n]²) caused by the micro vibration of the heartbeat. The lower stage of FIG. 19 illustrates peak times of the amplitude P[n]=√(mi[n]²+mq[n]²) caused by the micro vibration of the heartbeat. Here, the processing illustrated in FIG. 8 and FIG. 9 is performed.

The left section of FIG. 20 illustrates the weight of the heartbeat interval τₐ. The middle section of FIG. 20 illustrates the weight of the heartbeat interval τ_{b}. The right section of FIG. 20 illustrates the weight of the heartbeat interval τ_{c}. Here, in the heartbeat interval of 0<τₐ<50 and the heartbeat interval of 150<τ_{c}<200, the upper limit and the lower limit of the heartbeat interval are exceeded and the heartbeat interval is ignored. Then, at the heartbeat interval τₐ≈90 and the heartbeat interval τ_{b}≈90, clusters of the weight of the heartbeat interval are extracted. Accordingly, 61 bpm can be calculated as the heart rate. Note that when the weight is low, the detection result may be ignored, or the detection result may be warned. In addition, when the cluster is extracted, K-means clustering, DBSCAN, or the like may be applied.

In the lower section of FIG. 20, different from the left section, the middle section, and the right section of FIG. 20, the weights of the heartbeat intervals τₐ, τ_{b}, and τ_{c} are not illustrated separately, but the weights of the heartbeat intervals τₐ, τ_{b}, and τ_{c} are synthesized and illustrated. Here, clusters are extracted from this two-dimensional data, and outliers are removed. For the clusters, a weighted average of the heartbeat interval is calculated, making the accuracy of the heart rate higher.

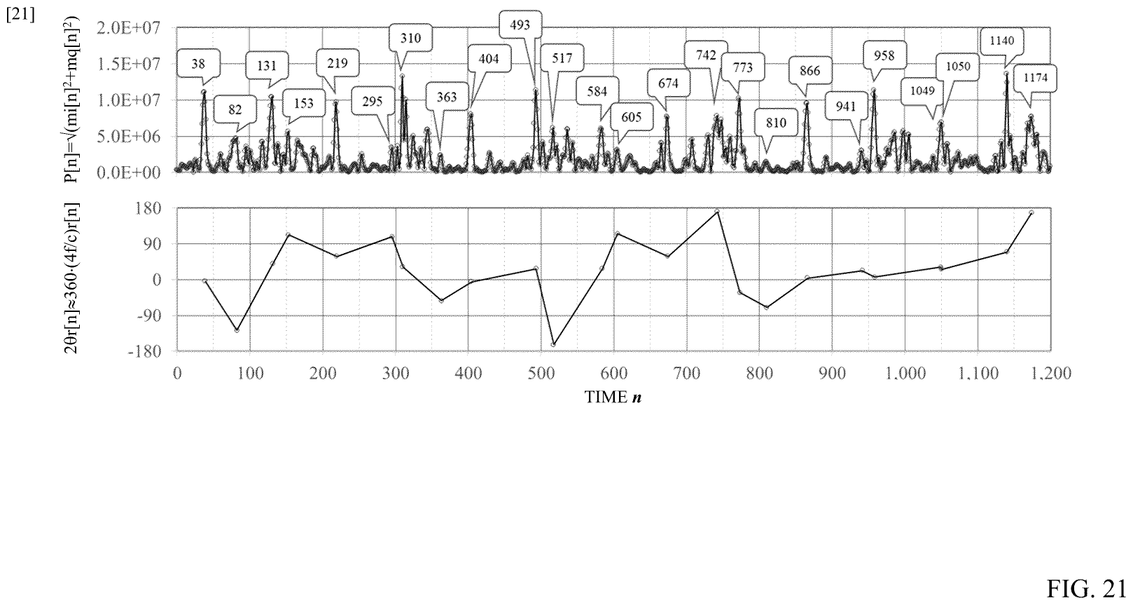

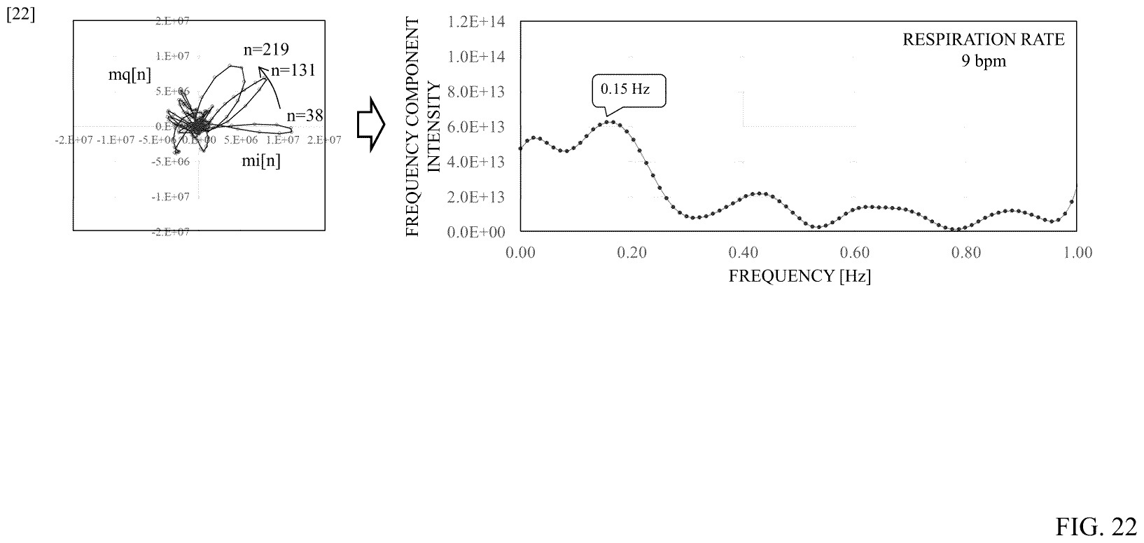

FIG. 21 and FIG. 22 illustrate results of the respiration detection processing of this disclosure. The upper stage of FIG. 21 illustrates the temporal change of the amplitude P[n]=√(mi[n]²+mq[n]²) caused by the micro vibration of the heartbeat. The lower stage of FIG. 21 illustrates a respiration phase change 2θᵣ[n]−π=360·(4f/c)r[n](r[n] is a distance) of the amplitude peaks. Here, the processing illustrated in FIG. 15 and FIG. 18 is performed.

The left section of FIG. 22 illustrates a constellation of the amplitude peaks. The right section of FIG. 22 illustrates a frequency conversion of the respiration phase change 2θᵣ[n]−π. Here, a monotonous change of the respiration phase change 2θᵣ[n]−π is detected from n=38 through n=131 to n=219. Then, the maximum peak frequency of the frequency conversion is calculated at the frequency component=0.15 Hz of the frequency conversion. Accordingly, 9 bpm can be calculated as the respiration rate.

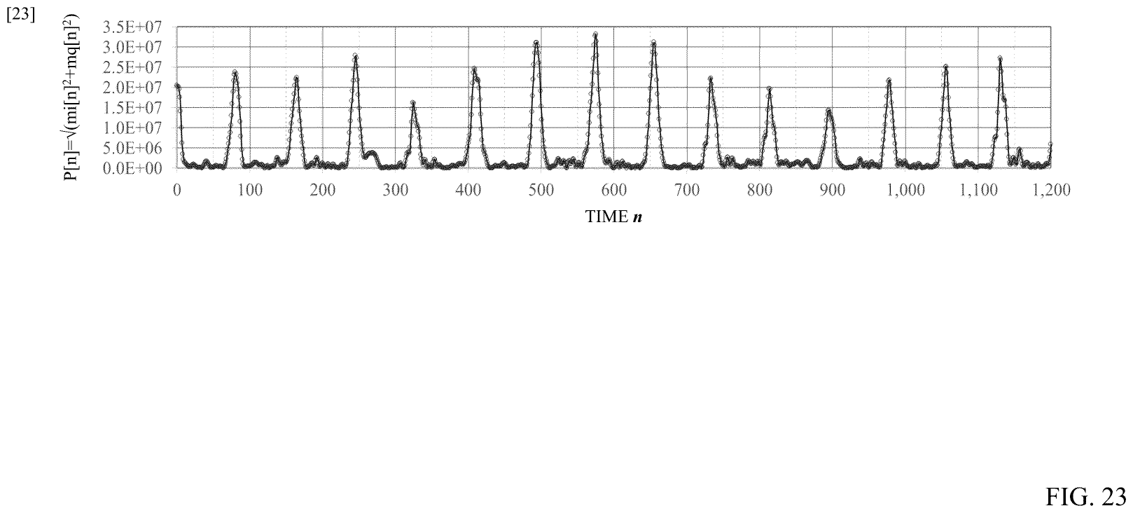

FIG. 23 also illustrates a result of the respiration detection processing of this disclosure. FIG. 23 illustrates the temporal change of the amplitude P[n]=√(mi[n]²+mq[n]²) caused by the micro vibration of the heartbeat. Here, the amplitude P[n] caused by the micro vibration of the heartbeat forms peaks very regularly. In this case, even when only the respiration amplitude change |A|² caused by the micro vibration of the respiration is extracted, the respiration rate can be calculated with high accuracy to some extent. However, the amplitude P[n] caused by the micro vibration of the heartbeat does not necessarily form peaks regularly. Even in this case, by extracting the respiration phase change 2θᵣ−π caused by the micro vibration of the respiration, the respiration rate can be calculated with high accuracy regardless of a situation.

### INDUSTRIAL APPLICABILITY

The heartbeat and respiration detection device and the heartbeat and respiration detection program of this disclosure can calculate a heart rate and a respiration rate while enabling the alleviation of burdens on nurses and infection risk reduction based on a radar signal (an ultrasound signal may be included) reflecting off a body surface.

### REFERENCE SIGNS LIST

S Spectrogram
B Band-pass filter result
P Patient
R Radar transmission/reception device, ultrasound transmission/reception device
M Heartbeat and respiration detection device
Wa, Wb, Wc, Wd Heartbeat observation time window
W0, W1, W2, W3 Peak detection time window
W Peak detection time window
1 Heartbeat component extraction unit
2 Heartbeat component multiplication unit
3 Heartbeat peak extraction unit
4 Heart rate calculation unit
5 Respiration phase extraction unit
6 Respiration rate calculation unit

1. A heartbeat and respiration detection device comprising:
a heartbeat component extraction unit that extracts a frequency component caused by a micro vibration of heartbeat from a radar signal or an ultrasound signal reflecting off a body surface over a predefined heartbeat observation time window;
a heartbeat peak extraction unit that extracts an amplitude peak from the frequency component caused by the micro vibration of the heartbeat and extracts one characteristic micro vibration out of a plurality of characteristic micro vibrations in one heartbeat; and
a heart rate calculation unit that calculates a heart rate based on a time interval of the amplitude peaks or a count of the amplitude peaks extracted within a predetermined time.

2. The heartbeat and respiration detection device according to claim 1, wherein
the heartbeat component extraction unit extracts the frequency component caused by the micro vibration of the heartbeat over the predefined heartbeat observation time window including a plurality of characteristic micro vibrations in one heartbeat.

3. The heartbeat and respiration detection device according to claim 1, wherein
the heartbeat peak extraction unit extracts a maximum amplitude peak of the amplitude peaks from the frequency component caused by the micro vibration of the heartbeat in a predefined peak detection time window.

4. The heartbeat and respiration detection device according to claim 3, wherein
the heartbeat peak extraction unit moves the predefined peak detection time window so as to extract the maximum amplitude peak in a time domain excluding vicinities of both ends of the predefined peak detection time window.

5. The heartbeat and respiration detection device according to claim 3, wherein
the heart rate calculation unit calculates a heart rate based on a time interval of the maximum amplitude peaks extracted in an adjacent or non-adjacent predefined peak detection time window.

6. The heartbeat and respiration detection device according to claim 1, wherein
the heart rate calculation unit calculates an average heart rate with increasing a weight of a time interval of the amplitude peaks as an amplitude value of the amplitude peak increases.

7. The heartbeat and respiration detection device according to claim 1, wherein
the heart rate calculation unit calculates a heart rate based on clustering of two-dimensional data constituted of a time interval of the amplitude peaks and a weight of the time interval with increasing the weight of the time interval of the amplitude peaks as an amplitude value of the amplitude peak increases.

8. A heartbeat and respiration detection device comprising:
a heartbeat component multiplication unit that extracts positive and negative frequency components caused by a micro vibration of heartbeat from a radar signal or an ultrasound signal reflecting off a body surface and performs complex multiplication of the positive and negative frequency components;
a respiration phase extraction unit that extracts a respiration phase change caused by a micro vibration of respiration, with a heartbeat phase change caused by a micro vibration of heartbeat removed, from complex multiplication of the positive and negative frequency components; and
a respiration rate calculation unit that calculates a respiration rate based on a frequency component of the respiration phase change.

9. The heartbeat and respiration detection device according to claim 8, wherein
the respiration phase extraction unit extracts amplitude peaks caused by a micro vibration of heartbeat from complex multiplication of the positive and negative frequency components and extracts the respiration phase change at the amplitude peak while applying zero padding between the amplitude peaks without extracting the respiration phase change.

10. The heartbeat and respiration detection device according to claim 8, wherein
the respiration rate calculation unit calculates an average respiration rate with increasing a weight of a respiration rate as a maximum peak amplitude of the frequency component of the respiration phase change increases.

11. A heartbeat and respiration detection program storage medium readable by a computer for causing the computer to execute each processing step corresponding to each processing unit of the heartbeat and respiration detection device according to claim 1.

12. A heartbeat and respiration detection program storage medium by a computer for causing the computer to execute each processing step corresponding to each processing unit of the heartbeat and respiration detection device according to claim 8.
